(19) United States
(12) Patent Application Publication
(10) Pub. No.: US 2025/0124801 A1
(43) Pub. Date: Apr. 17, 2025

(54) BEHAVIOR CHANGE SYSTEM

(71) Applicant: Motiv8 Technologies, Inc., Palo Alto, CA (US)

(72) Inventors: Eugene H. Lee, Palo Alto, CA (US); David McKinnon Lawrence, Geyserville, CA (US); Khanderao Kand, San Jose, CA (US)

(21) Appl. No.: 19/000,623

(22) Filed: Dec. 23, 2024

Related U.S. Application Data
(63) Continuation-in-part of application No. 18/377,174, filed on Oct. 5, 2023, now abandoned.
Application No. 18/377,174 is a continuation of application No. 15/487,908, filed on Apr. 14, 2017, now abandoned.
(60) Provisional application No. 62/322,367, filed on Apr. 14, 2016.

(51) Int. Cl. G09B 5/06; G16B 5/00; G16H 10/60; G16H 20/70; G16H 40/63; G16H 50/20; G16H 50/50; G16H 50/70

(52) U.S. Cl. CPC G09B 5/06; G16B 5/00; G16H 10/60; G16H 20/70; G16H 40/63; G16H 50/20; G16H 50/50; G16H 50/70

(57) ABSTRACT

Systems and methods for controlling behavior change in a user. Systems can include a behavior change model management system and a behavior change facilitation system included as part of a behavior change platform. Methods can include selecting a behavior change model based on a behavior-specific behavior change phenotype of a user and applying the behavior change model to control behavior change in the user.

### CROSS-REFERENCE TO RELATED APPLICATIONS

This application is a continuation-in-part of U.S. patent application Ser. No. 18/377,174 filed Apr. 14, 2017, which is a continuation of U.S. patent application Ser. No. 15/487,908 filed Apr. 14, 2017, which claims priority to U.S. Provisional Patent Application Ser. No. 62/322,367 filed Apr. 14, 2016, each of which is incorporated herein by reference.

### BACKGROUND

There are multiple approaches to facilitating changes in behavior to improve health and well-being. However, an approach that works for one person may not work for others. What is needed is a system that can select and provide a personalized approach to facilitating behavior change that is tailored to the qualities and needs of each individual.

### SUMMARY

The following implementations and aspects thereof are described and illustrated in conjunction with systems, tools, and methods that are meant to be exemplary and illustrative, not necessarily limiting in scope.

In various implementations, a method includes receiving user data for a user, the user data including a desired behavior change in the user; monitoring and transmitting, by one or monitoring devices of the user, activity data of the user, location data of the user, and data for one or more of locations, establishments, and entities in proximity to the user; the activity data of the user, the location data of the user, and the data for the one or more of locations, establishments, and entities in proximity to the user from the one or more monitoring devices of the user; determining content to produce for the user, the content being determined based on the desired behavior change in the user, the activity data of the user, the location data of the user, and the data for the one or more of locations, establishments, and entities in proximity to the user; generating content data to be used to produce the content; providing the content data to a device of the user for use in producing the content in order to facilitate the behavior change in the user.

In various implementations, a method includes identifying an experimental population for purposes of testing a behavior change model; applying the behavior change model to the experimental population; and updating the behavior change model according to behavior change outcomes observed through application of the behavior change model to the experimental population.

In various implementations, a system includes engines and/or means configured to perform the methods described above.

### BRIEF DESCRIPTION OF THE DRAWINGS

FIG. 1 depicts a diagram of an example of a system for initiating, encouraging, and sustaining behavior changes of users through a behavior change platform.

FIG. 2 depicts a flowchart of an example of a method for managing behavior changes of a user.

FIG. 3 depicts a flowchart of an example of a method for initiating, encouraging, and sustaining behavior changes of a user based on context.

FIG. 4 depicts a diagram of an example of a flow for classifying behavior change models based on outcomes to different user populations, then selecting behavior change models to apply for a user and then updating the behavior change models based on outcomes of behavior change of users grouped into the behavior change models.

FIG. 5 depicts a diagram of an example behavior change model management system.

FIG. 6 depicts a flowchart of an example of a method for maintaining a behavior change model for use in facilitating behavior changes in users.

FIG. 7 depicts a diagram of an example behavior change facilitation system.

FIG. 8 depicts a flowchart of an example of a method of applying a behavior change model for purposes of facilitating behavior change in a user.

FIG. 9 depicts a diagram of a behavior change content production management system.

FIG. 10 depicts a flowchart of an example of a method for controlling production of content to facilitate behavior changes in a user through application of a behavior change model selected based on the user's phenotype.

FIGS. 11A and 11B depict a flowchart of an example of a method for facilitating behavior changes in a user through content generation, activity and context monitoring devices, and nudge devices.

### DETAILED DESCRIPTION

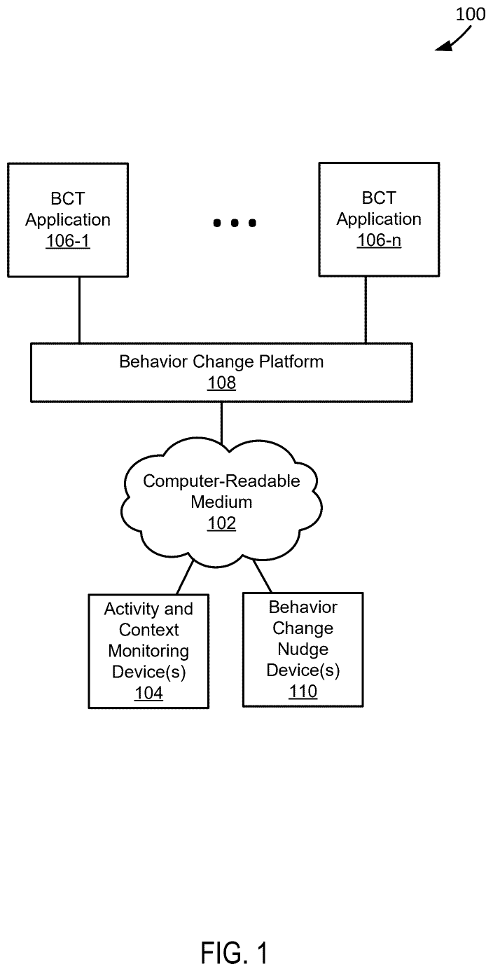

FIG. 1 depicts a diagram 100 of an example of a system for initiating, encouraging, and sustaining behavior changes of users through a behavior change platform. The system of the example of FIG. 1 includes a computer-readable medium 102, activity and context monitoring device(s) 104, behavior change technique (BCT) applications 106-1 to 106-n (hereinafter referred to as “BCT applications 106”), a behavior change platform 108, and behavior change nudge device(s) 110.

The computer-readable medium 102 and other computer readable mediums discussed in this paper are intended to include all mediums that are statutory (e.g., in the United States, under 35 U.S.C. 101), and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware.

The computer-readable medium 102 and other computer readable mediums discussed in this paper are intended to represent a variety of potentially applicable technologies. For example, the computer-readable medium 102 can be used to form a network or part of a network. Where two components are co-located on a device, the computer-readable medium 102 can include a bus or other data conduit or plane. Where a first component is co-located on one device and a second component is located on a different device, the computer-readable medium 102 can include a wireless or wired back-end network or LAN. The computer-readable medium 102 can also encompass a relevant portion of a WAN or other network, if applicable.

The computer-readable medium 102, the activity and context monitoring device(s) 104, the BCT applications 106, the behavior change platform 108, and other applicable systems or devices described in this paper can be implemented as a computer system or parts of a computer system or a plurality of computer systems. A computer system, as used in this paper, is intended to be construed broadly. In general, a computer system will include a processor, memory, non-volatile storage, and an interface. A typical computer system will usually include at least a processor, memory, and a device (e.g., a bus) coupling the memory to the processor. The processor can be, for example, a general-purpose central processing unit (CPU), such as a microprocessor, or a special-purpose processor, such as a microcontroller.

The memory can include, by way of example but not limitation, random access memory (RAM), such as dynamic RAM (DRAM) and static RAM (SRAM). The memory can be local, remote, or distributed. The bus can also couple the processor to non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software on the computer system. The non-volatile storage can be local, remote, or distributed. The non-volatile storage is optional because systems can be created with all applicable data available in memory.

Software is typically stored in the non-volatile storage. Indeed, for large programs, it may not even be possible to store the entire program in the memory. Nevertheless, it should be understood that for software to run, if necessary, it is moved to a computer-readable location appropriate for processing, and for illustrative purposes, that location is referred to as the memory in this paper. Even when software is moved to the memory for execution, the processor will typically make use of hardware registers to store values associated with the software, and local cache that, ideally, serves to speed up execution. As used herein, a software program is assumed to be stored at an applicable known or convenient location (from non-volatile storage to hardware registers) when the software program is referred to as “implemented in a computer-readable storage medium.” A processor is considered to be “configured to execute a program” when at least one value associated with the program is stored in a register readable by the processor.

In one example of operation, a computer system can be controlled by operating system software, which is a software program that includes a file management system, such as a disk operating system. Examples of operating system software with associated file management system software include the WINDOWS® operating system and the LINUX® operating system. The file management system is typically stored in the non-volatile storage and causes the processor to execute the various acts required by the operating system to input and output data and to store data in the memory, including storing files on the non-volatile storage.

The bus can also couple the processor to the interface. The interface can include one or more input and/or output (I/O) devices. Depending upon implementation-specific or other considerations, the I/O devices can include, by way of example but not limitation, a keyboard, a mouse or other pointing device, disk drives, printers, a scanner, and other I/O devices, including a display device. The display device can include, by way of example but not limitation, a cathode ray tube (CRT), liquid crystal display (LCD), or some other applicable known or convenient display device. The interface can include one or more of a modem or network interface. It will be appreciated that a modem or network interface can be considered to be part of the computer system. The interface can include an analog modem, ISDN modem, cable modem, token ring interface, satellite transmission interface (e.g. “direct PC”), or other interfaces for coupling a computer system to other computer systems. Interfaces enable computer systems and other devices to be coupled together in a network.

The computer systems can be compatible with or implemented as part of or through a cloud-based computing system. As used in this paper, a cloud-based computing system is a system that provides virtualized computing resources, software and/or information to end user devices. The computing resources, software and/or information can be virtualized by maintaining centralized services and resources that the edge devices can access over a communication interface, such as a network. “Cloud” may be a marketing term and for the purposes of this paper can include any of the networks described herein. The cloud-based computing system can involve a subscription for services or use a utility pricing model. Users can access the protocols of the cloud-based computing system through a web browser or other container application located on their end user device.

A computer system can be implemented as an engine, as part of an engine or through multiple engines. As used in this paper, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors or a first engine and a second engine can share one or more processors with one another or other engines. Depending upon implementation-specific or other considerations, an engine can be centralized or its functionality distributed. An engine can include hardware, firmware, or software embodied in a computer-readable medium for execution by the processor. That is, the engine includes hardware. The processor transforms data into new data using implemented data structures and methods, such as is described with reference to the FIGS. in this paper.

The engines described in this paper, or the engines through which the systems and devices described in this paper can be implemented, can be cloud-based engines. As used in this paper, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used in this paper, datastores are intended to include repositories having any applicable organization of data, including tables, comma-separated values (CSV) files, traditional databases (e.g., a structured query language (SQL) database), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described in this paper.

Datastores can include data structures. As used in this paper, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described in this paper, can be cloud-based datastores. A cloud-based datastore is a datastore that is compatible with cloud-based computing systems and engines.

The activity and context monitoring device(s) 104 are intended to represent devices that function to generate, transmit, (and potentially receive), data for use in managing behavior changes of users. Data generated and transmitted by the activity and context monitoring device(s) 104 can include either or both activity and context data. Activity data includes data describing activity and health of a user. For example, activity data can include vitals of a user, whether a user took their medication, and a distance a user has walked. Context data includes data describing a context associated with a user. A context associated with a user can include applicable circumstances associated with the user at any given time. For example, a context associated with a user can include a location of a user at a specific time and activities a user is undertaking or will undertake at a specific time. Additionally, a context associated with a user can include applicable parameters describing a current state or environment of a user. For example, context can include an environment a user is in, a time of day a user is currently at, calendar entries of a user, weather, and locations, establishments, or entities in proximity to a user.

Behavior changes of a user include changes to a person's daily activities, habits, or other applicable parameters describing behaviors of a person. Behavior changes of users can include changes of a person's behaviors with respect to their health. For example, behavior changes can include a person consuming fewer calories in a day. In another example, behavior changes can include a person properly taking their medication. In a specific implementation, the activity and context monitoring device(s) 104 can include thin clients or ultra-thin clients. For example, the activity and context monitoring device(s) 104 can include a smart phone. In various implementations, the activity and context monitoring device(s) 104 can include a wireless network interface. For example, the activity and context monitoring device(s) 104 can include an IEEE 802.11-compatible network interface through which the activity and context monitoring device(s) 104 can at least send (and perhaps receive) data wirelessly thorough a wireless LAN. In another example, the activity and context monitoring device(s) 104 can include a cellular network interface through which the activity and context monitoring device(s) 104 can at least send (and perhaps receive) data wirelessly through a cellular network.

In a specific implementation, the activity and context monitoring device(s) 104 functions as a wearable or is coupled to a wearable. In various implementations, a wearable can include a device with applicable sensors or measurement mechanisms for measuring vital statistics, movements, or environmental factors of a user. For example, a wearable can include accelerometers and orientation sensors for determining a number of steps taken by a user. In another example, a wearable can include a heart rate monitor for monitoring a heart rate of a user. In various implementations, data generated by the activity and context monitoring device(s) 104 functioning as a wearable or a wearable coupled to the activity and context monitoring device(s) 104 can be sent by the activity and context monitoring device(s) 104 for use in initiating, encouraging, or sustaining behavior changes of a user. For example, a heart rate reading generated by a heart rate monitor coupled to the activity and context monitoring device(s) 104 can be sent from the activity and context monitoring device(s) 104 to ensure that a user is properly taking their blood pressure medication.

The BCT applications 106 are intended to represent applications that function to receive activity and/or context data for a specific user or type of user and provide behavior change nudges or dynamic escalations to a user or applicable party. Each of the BCT applications 106 can include a single BCT or multiple BCTs. In a health context, the BCT applications 106 can be used, at least in part, to help monitor activity of a user and/or initiate, encourage, and/or sustain healthy behavior change in the user. While the term health is used throughout this paper, the functionalities and processes described in this paper can be applied to behavior changes related to fitness, advertising, marketing, and education. Examples of health related applications include applications for monitoring patient vitals or measuring user performance statistics. In various implementations, BCT applications 106 can include applications that receive data from an activity or context monitoring device, e.g. a client device of a user and/or a wearable of a user. For example, health related applications can include an application for receiving data indicating a number of steps a user walked during a day. Depending upon implementation-specific or other considerations, BCT applications 106 can be third party applications. For example, BCT applications 106 can include a Fitbit® Application.

The behavior change platform 108 is intended to represent a platform that functions to facilitate behavior changes of users. In facilitating behavior changes of users, the behavior change platform 108 can initiate, encourage, and sustain behavior changes of users. In a specific implementation, the behavior change platform 108 functions as a platform to initiate, encourage, and sustain behavior change using the BCT applications 106. The behavior change platform 108 and at least a portion of its functionalities can be integrated as part of an application including a BCT application, e.g. a fitness application.

In a specific implementation, in facilitating behavior changes of users, the behavior changes platform 108 functions to facilitate communication with users. The behavior change platform 108 can facilitate communication with users through, e.g. health- or fitness-related applications. For example, the behavior change platform 108 can instruct a health improvement application to send a notification, e.g. a contextual notification, to a user who is using the health improvement application, for purposes of changing behaviors of the user. Additionally, the behavior change platform 108 can communicate directly with users. For example, the behavior change platform 108 can directly send a notification, e.g. a contextual nudge, to a device utilized by a user for purposes of changing behaviors of the user. Notifications sent by the behavior change platform 108, either directly or through other applications, can vary depending upon characteristics of the user and current context.

In a specific implementation, the behavior change platform 108 functions to receive data used in facilitating behavior changes, e.g. improving, encouraging, and sustaining behavior changes of users from either or both of BCT applications 106 and activity and context monitoring device(s) 104. Specifically, the behavior change platform 108 can receive either or both activity and context data. For example, the behavior change platform 108 can receive activity data indicating a number of steps a user has taken by analyzing data from the activity and context monitoring device(s) 104, such as from a wearable device of the user, or from the BCT applications 106 for tracking steps, which utilize data from the activity and context monitoring device(s) 104. In another example, the behavior change platform 108 can receive context data indicating a current location of a user and surrounding restaurants at the current location of the user.

Initiating, encouraging, and sustaining behavior changes includes one or an applicable combination of initiating behavior changes in users, encouraging behavior changes in users, and sustaining behavior changes in users. For example, initiating, encouraging, and sustaining behavior changes includes initiating an interest in a user to lose weight, encouraging a user to lose weight, and sustaining a user's weight loss. In another example, initiating, encouraging, and sustaining behavior changes including initiating an interest in a user to cat healthy, encouraging a user to cat healthy, and sustaining a user's healthy eating.

In a specific implementation, the behavior change platform 108 functions to facilitate behavior changes in users according to behavior change models. Behavior change models are models that show applicable information related to behaviors of users. Examples of information included as part of behavior change models include behaviors exhibited by users, changes in behaviors of users, target changes in behaviors of users, undesirable changes in behaviors of users, communications with users corresponding to changes in behaviors of users, including both desirable and undesirable changes, types and/or content of communications/notifications sent to users corresponding to changes in behaviors of users, behavior-specific behavior change phenotypes in which users are grouped into according to either or both behavior-specific behavior change phenotype variables and dynamic state responsiveness phenotype variables, BCTs (which can include motivational rules), BCTs for managing communication with users for eliciting behavior changes, psychological and cognitive factors describing behaviors of user, psychological and cognitive factors and techniques for predicting and influencing behaviors of user, devices and wearables utilized by users, and dynamic state responsiveness phenotype variables. For example, a behavior change model can specify to send a notification at a specific time reminding a user who has diabetes to take their medicine. Example behavior-specific behavior change phenotype variables include demographic, geographic, psychographic, behavioristic, and personality trait variables. Example personality trait variables include openness to experiences, conscientiousness, extraversion, agreeableness, and neuroticism. For example, values of behavior-specific behavior change phenotype variables can include demographic information, such as ethnicity and age. In another example, values of behavior-specific behavior change phenotype variables can include activities a user likes performing, wants to perform, or has performed. In still another example, values of behavior-specific behavior change phenotype variables can include diagnosis or illnesses of a user.

In a specific implementation, BCTs for managing communications with a user are context specific. More specifically, BCTs for managing communications with a user, as included in a behavior change model, are context specific. In being context specific, BCTs, including motivational rules, can be applied according to a context associated with a user. The behavior change platform 108 can facilitate communicating with a user according to motivational rules selected based on a context associated with a user. For example, if a context associated with a user indicates that the user is in close proximity to a vegan restaurant, then the behavior change platform 108 can select motivational rules to apply when users are close to a vegan restaurant. Additionally, the behavior change platform 108 can facilitate communicating with a user by following motivational rules based on a context associated with the user. For example, if a motivational rule indicates to alert a user when they are in close proximity to a specific gym, and a context associated with the user indicates the user is in close proximity to the specific gym, then the behavior change platform 108 can facilitate alerting the user they are in close proximity to the specific gym.

In some embodiments, the behavior change platform 108 includes and/or use a global positioning system (GPS) and/or database of pre-identified locations to identify locations (e.g., a vegan restaurant) and/or determine whether one or more users are in a close proximity (e.g., within a threshold distance or radius, such as 10 meters, 100 meters, 1000 meters, 1 mile, etc.) to a location (e.g., vegan restaurant).

In a specific implementation, the behavior change platform 108 functions to select a behavior change model for a user, or otherwise group the user into the behavior change model. A behavior change model selected for a user can be utilized to or otherwise guide the behavior change platform 108 in facilitating behavior changes in the user. For example, the behavior change platform 108 can control communicating with a user according to motivational rules, e.g. BCTs, included in a behavior change model selected for the user. The behavior change platform 108 can select a behavior change model for a user according to behavior-specific behavior change phenotype of a user. Further the behavior change platform 108 can select a behavior change model for a user according to one or a combination of a behavior-specific behavior change phenotype variable values of the user, dynamic state responsiveness phenotype variables of the user, and a context associated with the user. The behavior change platform 108 can group users into behavior change models based on data received from a user or other data source. For example, the behavior change platform 108 can receive data directly from a user indicating the user has been diagnosed with diabetes and subsequently group the user to a behavior change model based on the data received from the user. Further depending upon implementation-specific or other considerations, the behavior change platform 108 can group users into behavior change models based on data received through a health related application. For example, the behavior change platform 108 can receive data indicating a user has been diagnosed with diabetes from a health related application utilized by a user and subsequently group the user to a behavior change model based on the data received from the health application.

In a specific implementation, the behavior change platform 108 functions to maintain behavior change models for use in facilitating behavior changes in users. In maintaining behavior change models, the behavior change platform 108 can create new behavior change models or update or modify already existing behavior change models. For example, the behavior change platform 108 can change BCTs included as part of a behavior change model. Further, in maintaining a behavior change model the behavior change platform 108 can define behavior-specific behavior change phenotype variables that form a behavior-specific behavior change phenotype into which users can be segmented. For example, the behavior change platform can define an age and gender for a behavior-specific behavior change phenotype of a behavior change model.

In a specific implementation the behavior change platform 108 functions to incorporate dynamic state responsiveness phenotype variables into either or both a behavior change model and a behavior-specific behavior change phenotype. Dynamic state responsiveness phenotype variables are behavior-specific behavior change phenotype variables capable of changing at a given time. For example, a behavior-specific behavior change phenotype can include a dynamic state responsiveness phenotype variable including whether a user's illness is cured. Dynamic state responsiveness phenotype variables can change based on a given context. For example, a value of a dynamic state responsiveness phenotype variable can include that a user had a falling out with their mother when they were younger because they were engaging in unhealthy behavior, which can have an impact on how a user responds to contextual nudges related to healthy behavior. In another example, a value of a dynamic state responsiveness phenotype variable can include whether a person is currently low on the personality attribute of agreeableness. Further in the example, a person who is currently low on the personality attribute of agreeableness will tend to have negatively reinforcing self-doubt if they miss their current short term goals, and thus will react negatively to aggressive exhortations.

In a specific implementation, behavior change phenotypes are variable depending upon a specific behavior targeted for change. For example, a user who is responsive to a specific type of contextual nudge to lose weight, such as an aggressive message that they need to stay on track, might be responsive to a different type of contextual nudge to take medications, such as a nurturing nudge expressing concern about health. As another example, a user who was athletic when young might have self-confidence about increasing physical activity, but also has teenage children at home and thus may struggle more with healthy eating because of the unhealthy snacks in the house. For this reason, throughout this paper, the behavior change phenotypes are often referred to as behavior-specific behavior change phenotypes.

In a specific implementation, the behavior change platform 108 functions to incorporate context related to a user for purposes of facilitating behavior change in the user. The behavior change platform 108 can use received context data in facilitating behavior change in a user based on context. For example, if a user is currently in a fast food restaurant, the behavior change platform 108 can generate messages related to what menu items are healthy, how the user has a goal of running a 5K, or whatever other message can become important in view of the current location of the user. Other contextual nudges can be related to calendar items, such as if the user has an hour free after lunch, the system can instruct a health app to nudge the user that it's a good time to take a walk.

In a specific implementation, the behavior change platform 108 functions to determine contexts of users for use in incorporating context into facilitating behavior changes of the users. The behavior change platform 108 can determine a context associated with a user based on data, e.g. context data, received from a health related application, user device sensors, and/or directly from the user. For example, the behavior change platform 108 can determine a location of a user based on data received from a client device of the user. In another example, the behavior change platform 108 can determine that a user has not refilled their diabetes medication based on updated data from an Electronic Health Record or health insurance claims system. In facilitating behavior changes of users based on context, the behavior change platform 108 can use a determined context associated with a user to provide contextual notifications to the user.

In a specific implementation, the behavior change platform 108 is configured to facilitate behavior changes in a user utilizing a function that can be defined as function ƒ(β, φ, Δ). The function shows that communication can be initiated, e.g. a nudge, with users or people associated with a user as a function of values of behavior-specific behavior change phenotype variables β of a user, values of dynamic state responsive phenotype variables φ of the user, and a context Δ of the user. All users can be categorized as having a behavior-specific behavior change phenotype with an accuracy that depends upon the data points available for the user. The users can also be characterized as having a dynamic state responsiveness phenotype that can result in an adjustment of dynamic state responsiveness variable values in certain contexts. The users can also be characterized according to a specific context, such as location, proximity to certain harmful or helpful stimuli, recent performance history, and weather, to name a few.

In a specific implementation, by utilizing a function to facilitate changes in users dependent on either or both contexts and dynamic state responsiveness phenotype variables, the users grouped into a behavior change model, or who otherwise have had the behavior change model selected for them, can still have different behavior-specific behavior change phenotypes. For example, users grouped into a behavior change model can have shared behavior-specific behavior change phenotype variable values and different behavior-specific behavior change phenotype variable values. In another example, users grouped into a behavior change model can have the same behavior-specific behavior change phenotype variable values, but different dynamic state responsiveness phenotype variable values. A behavior change model can specify to apply different BCTs or motivational rules to different users grouped into the behavior change model. For example, based on differences in values of behavior-specific behavior change phenotype variables or dynamic state responsiveness phenotype variables of users grouped in the same behavior change model, different BCTs or motivational rules can be applied for facilitating behavior change in the users.

In a specific implementation, the behavior change platform 108 functions to maintain behavior change models according to behavior-specific behavior change phenotype variables. The behavior change platform 108 can create a behavior change model for a group of users defined by behavior-specific behavior change phenotype variables. For example, the behavior change platform 108 can create a behavior change model for users who are 35-year-old males living within a specific state, or for introverted 65-year-old women with Type 2 Diabetes who quit smoking but are obese. Additionally, the behavior change platform 108 can update behavior change models according to behavior-specific behavior change phenotype variables. For example, the behavior change platform 108 can split a behavior change model into two separate behavior change models based on a behavior-specific behavior change phenotypes variable. For example, the behavior change platform can split a behavior change model for introverted 65-year-old women with Type 2 Diabetes who quit smoking but are obese into 2 behavior-specific behavior change models including one for increasing physical activity and another for improving diet. In another example, the behavior change platform 108 can split a behavior change model representing males who use a specific device, into two behavior change models representing males who use a specific device in two different geographic regions.

In a specific implementation, the behavior change platform 108 functions to dynamically maintain behavior change models. For example, the behavior change platform 108 can update behavior change models as behaviors of users progress towards or regress from target behavior changes of the users. In another example, if a user changes their behavior, then the behavior change platform 108 can update a behavior change model representing the user to indicate the change in behavior of the user. In dynamically maintaining behavior change models, the behavior change platform 108 can update or change BCTs based on performance of users in response to communications with the users. For example, if a user changes their behaviors to a desired outcome based on specific notifications being sent to the user, then the behavior change platform 108 can update BCTs of a behavior change model in which a user is grouped to indicate sending of the specific notifications to the user to achieve desired behavior changes. Deviations from the behavior-specific behavior change phenotype in a given context can be recorded as dynamic state responsiveness phenotype variables.

In a specific implementation, the behavior change platform 108 functions to gather user data for use in managing behavior changes of the user. User data gathered by the behavior change platform 108 includes applicable data describing attributes of users for use in managing behavior changes of the user. User data can include either or both activity and context data of a user. For example, user data can include a user's vital statistics, illnesses or diseases a user is diagnosed with, behaviors exhibited by a user, a user's activities, represented as part of user activity data, and values of behavior-specific behavior change phenotype variables, e.g. age, sex, regions associated with a user. The behavior change platform 108 can utilize gathered user data of a user to group the user into one or more behavior change models and apply the behavior change models for managing behavior changes of the user. For example, behavior change platform 108 can gather user data from an applicable source, e.g. a health care provider. In another example, the behavior change platform 108 can gather user data from a health related application utilized by a user and/or from the user directly.

In a specific implementation, the behavior change platform 108 functions to group a user into a behavior change model based on likelihood of success in achieving desired behavior changes. For example, based on values of behavior-specific behavior change phenotype variables and/or user data of a user, the behavior change platform 108 can group a user into a behavior change model likely to cause the greatest behavior change in the user. The behavior change platform 108 can use machine learning in grouping users into behavior change models based on likelihood of success in achieving desired behavior changes. For example, if over time it is shown that males within a certain demographic are likely to achieve the greatest desired behavior changes when grouped into a specific behavior change model, then the behavior change platform 108 can learn to group males within the certain demographic into the specific behavior change model.

In a specific implementation, the behavior change platform 108 functions to determine a desired behavior change or changes for a user. The behavior change platform 108 can determine desired behavior changes for a user using either or both values of behavior-specific behavior change phenotype variables and user data of the user. For example, if values of behavior-specific behavior change phenotype variables indicate that a user is at risk of diabetes or prediabetic, then the behavior change platform 108 can determine that behavior changes for reducing chances of developing diabetes are recommended behavior changes of the user. The behavior change platform 108 can determine desired behavior changes for a user based on behavior change recommendation rules. Behavior change recommendation rules include rules for establishing target or desired behavior changes for a user based on characteristics of the user and can be included as part of a behavior change model. The behavior change platform 108 can either or both generate and update behavior change recommendation rules based on either or both experts and machine learning. For example, behavior change recommendation rules including initial recommendation rules for desired behavior changes causing weight loss can be established by a nutritionist expert. In another example, recommendation rules can be established through machine learning over time based on user success in achieving target behavior changes. A determined desired behavior change for a user can be used, at least in part, to group the user into a behavior change model for facilitating behavior changes of the user. Alternatively, a determined desired behavior change can be determined from a behavior change model selected for a user.

In a specific implementation, the behavior change platform 108 functions to facilitate behavior changes in users according to motivational rules. Motivational rules, included as part of BCTs, are rules specifying how to communicate with a user in eliciting behavior changes. For example, motivational rules can specify communication channels to use and what to communicate to users in eliciting behavior changes. Motivational rules can be specific to a group of users grouped according to behavior-specific behavior change phenotype values and be included as part of a behavior change model selected or capable of being selected for the group of users. For example, motivational rules can specify males who use smartphones respond better to bright message displays. Additionally, motivational rules can be specific to a user. For example, motivational rules can indicate that a specific user responds to verbal communication better than text based communication in eliciting behavior changes. Motivational rules can be context based. For example, motivational rules can specify that if a user is within the vicinity of a fast food restaurant, then a motivational message indicating the user's goals should be sent to the user. Additionally, the motivational rules can be further refined based on the dynamic state responsiveness phenotype variables, i.e. two users could be of the same behavior-specific behavior change phenotype, but have different dynamic state responsiveness phenotypes, and thus would benefit most from different nudges.

In a specific implementation, the behavior change platform 108 functions to maintain user profiles of users for managing behavior changes of the users. User profiles include applicable information related to users, e.g. a recommended behavior change of a user. For example, user profiles can include target behavior changes of users, behavior changes that have actually occurred in users, goals of users, ways in which to communicate with a user for purposes of eliciting behavior changes, and behavior change models users are grouped into according to one or a combination of behavior-specific behavior change phenotype variables, dynamic state responsiveness phenotype variables, and contexts of a user. For example, a user can specify that a goal of a user is to climb Machu Picchu. The behavior change platform 108 can use, at least in part, a user profile in communicating with a user to elicit behavior changes. For example, the behavior change platform 108 can send a message or cause a message to be sent reminding a user of their goal in climbing Machu Picchu. In another example, the behavior change platform 108 can determine that a user likes swimming and subsequently send a message to the user indicating that a pool is close to their current location. The behavior change platform 108 can maintain user profiles of users using data, e.g. user data, received directly from the users or from health related applications utilized by the users.

In a specific implementation, the behavior change platform 108 functions to provide an interface through which a caregiver/instructor/coach can view one or a combination of a user profile, behavior change models selected for a user, a user's behaviors, and changes made to a user's behaviors. For example, the behavior change platform 108 can provide an interface through which a caregiver can view behavior changes related to fitness of a diabetic patient in the attempts to improve the health of the patient. Additionally, the behavior change platform 108 can provide an interface through which a caregiver can view determined contexts of a patient. For example, if a patient has checked into a restaurant, then the behavior change platform 108 can provide an interface through which a caregiver can be informed of the context associated with the patient in checking into a restaurant.

In a specific implementation, the behavior change platform 108 functions to provide an interface through which a caregiver can recommend motivational rules, behavior change recommendation rules, or BCTs for use in facilitating behavior changes in a user. For example, if a caregiver observes that a user is not progressing towards their target health changes, then the caregiver can provide motivational rules, or BCTs to use in causing the user to progress towards their target health changes. The behavior change platform 108 can manage behavior changes of users according to motivational rules, behavior change recommendation rules, or BCTs provided by a caregiver through an interface to the behavior change platform 108. In providing interfaces through which caregivers can access the behavior change platform 108, caregivers can aid in providing BCTs or motivational rules to follow in guiding a user in changing behavior, as behavior change models are built up over time, e.g. through machine learning.

In a specific implementation, the behavior change platform 108 functions to manage dynamic escalation nudges. Dynamic escalation nudges include nudges or communications sent to people other than the user, such as friends, family, peers, or professional caregivers. In most cases these dynamic escalations are created to provide the user real-time human support. Additionally, the behavior change platform 108 can manage sending of dynamic escalation nudges to people other than a user without the knowledge of the user. Dynamic escalation nudges can be sent according to BCTs of a behavior change model in which a user is grouped. For example, if a behavior change model implicates an escalation, the behavior change platform 108 can send dynamic escalation nudges to relatives of a user if a user continues to neglect exercising asking them to send an encouraging text to the user. Alternatively, if the user has a negative self-image of their self-control and does not want to talk to friends or family about their eating habits, the system can dynamically escalate to another user with the same chronic disease or behavior change target.

The behavior change nudge device(s) 110 are intended to represent devices that function to receive and produce content for a user as part of facilitating behavior changes in the user. Content includes a message for production to a user in facilitating behavior changes in the user. For example, content can include a motivational spoken message. In another example, content can include a listing of the healthiest menu items at a restaurant. The behavior change nudge device(s) 110 can produce messages in a form capable of being perceived by a human. For example, the behavior change nudge device(s) 110 can include a speaker used to produce an auditory message. In another example, the behavior change nudge device(s) 110 can include a display for presenting a visual message to a user.

In a specific implementation, the behavior change nudge device(s) 110 function to receive contextual notifications, e.g. contextual nudges. Contextual notifications are notifications sent to a user as part of facilitating behavior changes in a user. The behavior change nudge device(s) can receive contextual notifications based on a function having the parameters of behavior-specific behavior change phenotype variables, dynamic state responsiveness phenotype variables, and context. Similarly, dynamic escalation nudges can be sent to behavior change nudge device(s) 110 of relevant parties, such as relatives, health care providers, teaches, coaches, or the like. An example of a contextual notification is that if a user is within a vicinity of an unhealthy restaurant, then a contextual notification can be sent to the user reminding the user of their goals of changing behaviors related to health.

In an example of operation of the example system shown in FIG. 1, the activity and context monitoring device(s) 104 function to send data related to user activity or context associated with a user to the BCT applications 106. Although FIG. 1 indicates the communications from the activity and context monitoring device(s) 104 is through the behavior change platform 108, the system can be implemented such that the data is sent from the activity and context monitoring device(s) 104 directly to the BCT applications 106 and some or all relevant data is provided to the behavior change platform 108 from the BCT applications 106, or the data can pass through the behavior change platform 108, which captures the relevant data. In the example of operation of the example system shown in FIG. 1, the behavior change platform 108 functions to initiate, encourage, and sustain behavior changes of the user of the activity context monitoring device(s) 104 using the data either directly by utilizing data from the user of the BCT applications 106 or indirectly via the BCT applications 106, which can be recommended, blocked, or otherwise controlled by the behavior change platform 108. The initiation, encouragement, and sustaining of behavior changes can be accomplished via contextual nudges or dynamic escalations provided to the behavior change nudge device(s) 110.

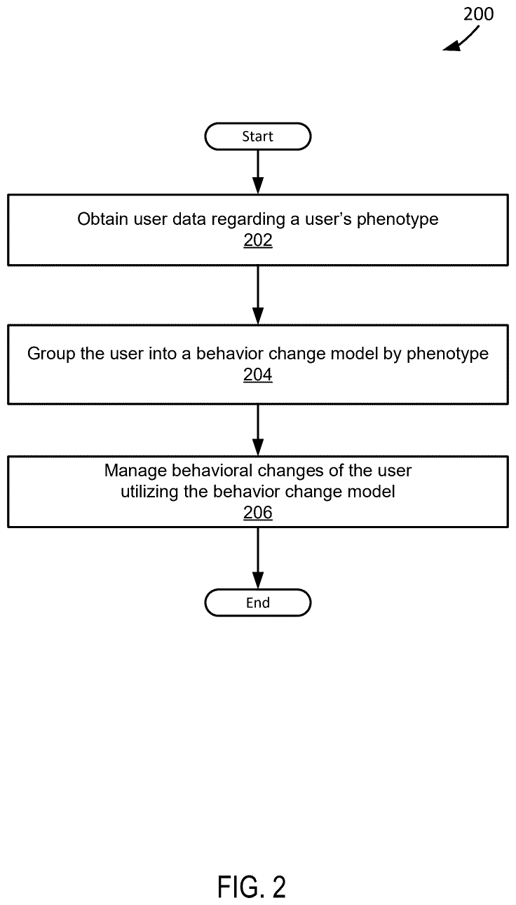

FIG. 2 depicts a flowchart 200 of an example of a method for managing behavior changes of a user. The flowchart 200 begins at module 202 where user data regarding a user's phenotype is obtained. User data can be gathered from a health care provider of a user. For example, user data can be gathered from a hospital providing health care to a user. Phenotype can include both behavior-specific behavior change phenotype and dynamic contextual responsiveness phenotype. An applicable platform for facilitating behavior change in a user, such as the behavior change platforms described in this paper, can obtain user data regarding a user's phenotype.

The flowchart 200 continues to module 204, where the user is grouped into a behavior change model based, at least in part, on phenotype. The user can be grouped into a behavior change model representing a group the user is grouped into based on behavior-specific behavior change phenotype variables applied to the user data. For example, if a user is an overweight 40 year old male living in a certain region in the country, then the user can be grouped into a behavior change model representing overweight males who are 40 years old and live in the certain region for a specific behavior, such as weight loss. The user can be grouped into a behavior change model based on likelihood of success in achieving desired behavior changes. For example, based on behavior-specific behavior change phenotype variables applied to the user data of the user, the user can be grouped into a behavior change model likely to cause the greatest behavior change in the user. Further depending upon implementation-specific or other considerations, the user can be grouped into a behavior change model based on determined target behavior changes determined from the user data using behavior change recommendation rules. For example, if the user data indicates the user is an overweight person who smokes, then it can be determined that a target behavior change is to quit smoking, and the user can be grouped into a behavior change model with a first target behavior change of quitting smoking. An applicable platform for facilitating behavior change in a user, such as the behavior change platforms described in this paper, can group the user into a behavior change model by phenotype.

The flowchart 200 continues to module 206, where behavior changes of the user are managed using, at least in part, the behavior change model appropriate for the phenotype of the user. Communications with the user can be managed according to BCTs included as part of the behavior change model, to elicit a change in behavior of the user. For example, if motivational rules indicate that users within the behavior change model respond best to auditory messages, then an auditory message to elicit a change in behavior can be played to the user. In various implementations, behavior changes can also be managed using a user profile of the user. For example, if a user profile of the user indicates a goal of the user, then this goal can be communicated to the user in attempting to elicit a change in behavior of the user. An applicable platform for facilitating behavior change in a user, such as the behavior change platforms described in this paper, can manage behavioral changes of the user utilizing the behavior change model.

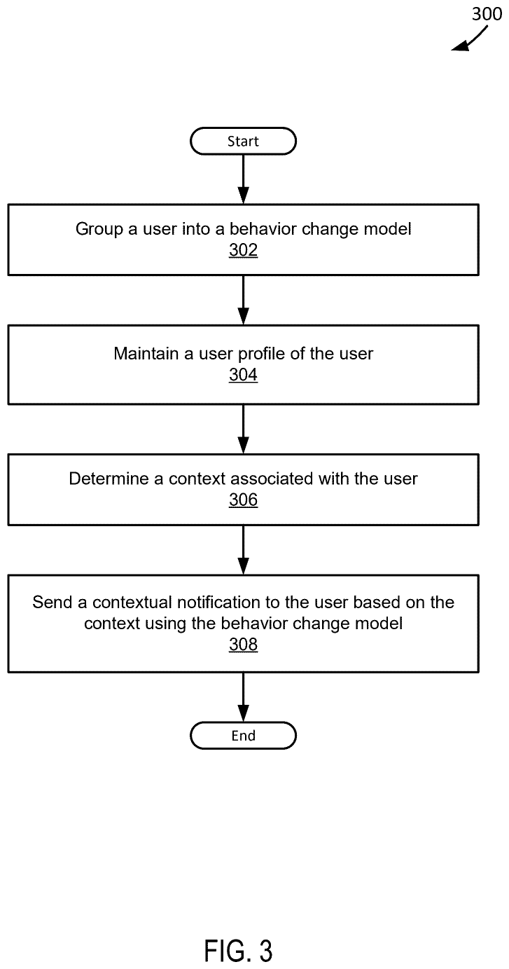

FIG. 3 depicts a flowchart 300 of an example of a method for initiating, encouraging, and sustaining behavior changes of a user based on context. The flowchart 300 begins at module 302, where a user is grouped into a behavior change model. A user can be grouped into a behavior change model representing a group the user is segmented into based on one or a combination of behavior-specific behavior change phenotype variables and dynamic state responsiveness phenotype variables applied to user data for the user. For example, if a user is an overweight 40 year old male living in a certain region in the country, then the user can be grouped into a behavior change model representing overweight males who are 40-years-old and live in the certain region. Additionally, a user can be grouped into a behavior change model based on likelihood of success in achieving desired behavior changes. For example, a user can be grouped into a behavior change model likely to cause the greatest behavior change in the user. Further, a user can be grouped into a behavior change model based on determined target behavior changes determined using behavior change recommendation rules. For example, if user data indicates a user is an overweight person who smokes, then it can be determined that a target behavior change is to quit smoking, and the user can be grouped into a behavior change model with a target behavior change of quitting smoking. An applicable platform for facilitating behavior change in a user, such as the behavior change platforms described in this paper, can group a user into a behavior change model.

The flowchart 300 continues to module 304, where a user profile of the user is maintained. A user profile of the user can be maintained using received user data of the user and the behavior change model into which the user is grouped. For example, a user profile of the user can specify health or fitness related goals of the user. In another example, if the user has a goal of running a 5K, then the user profile of the user can specify the user has the goal of running a 5K. Additionally a user profile of a user can be maintained according to phenotype variables of a user and potentially changing phenotype variables of a user, e.g. behavior-specific behavior change phenotype variables and dynamic state responsiveness phenotype variables. For example, a user profile of a user can be maintained to indicate changing phenotype variables reflecting changing behaviors of the user. An applicable platform for facilitating behavior change in a user, such as the behavior change platforms described in this paper, can maintain a user profile of the user.

The flowchart 300 continues to module 306, where a context associated with the user is determined. A context associated with the user can be determined based on data received from a health related application, e.g. a BCT application, and/or directly from the user. For example, a context associated with a user indicating a location of the user can be determined based on the location of a client device or a wearable of the user indicated by data received from either the client device or the wearable. An applicable platform for facilitating behavior change in a user, such as the behavior change platforms described in this paper, can determine a context associated with the user.

The flowchart 300 continues to module 308, where a contextual notification is sent to the user using the behavior change model. A contextual notification can be sent to the user based on motivational rules of BCTs included in the behavior change model. For example, if the behavior change model indicates to send a contextual notification to a user indicating that a user should take their medications at a specific time of day, and the context associated with the user indicates it is the specific time of day at the user's current location, then a contextual notification informing the user to take their medication can be sent to the user. A contextual notification can be sent to a user utilizing, at least in part, the user profile of the user. For example, a contextual notification can be sent to the user indicating a goal of the user, as included as part of the user profile.

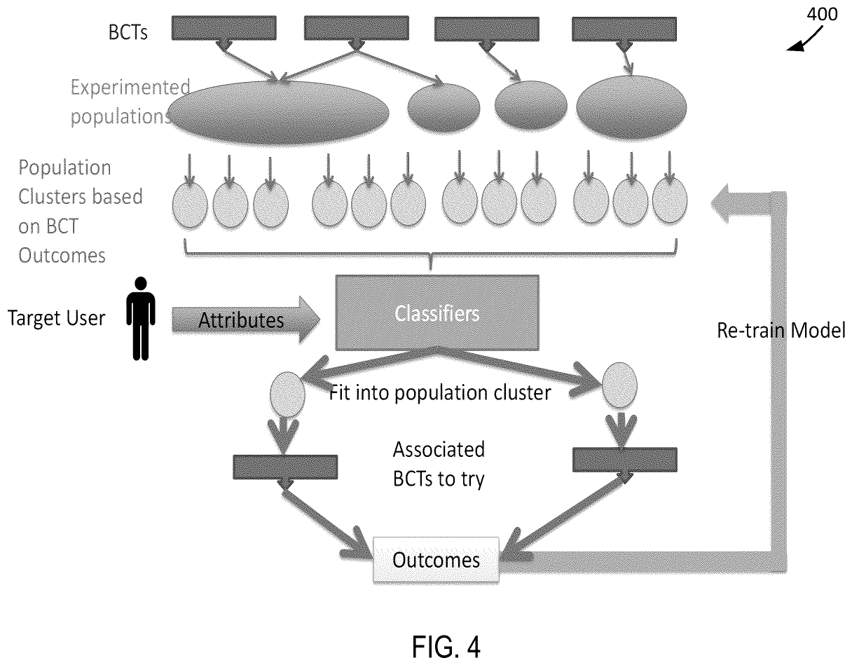

FIG. 4 depicts a diagram 400 of an example of a flow for classifying behavior change models based on outcomes to different user populations, then selecting behavior change models to apply for a user and then updating the behavior change models based on outcomes of behavior change of users grouped into the behavior change models. The flow begins where BCTs, or a taxonomy of BCTs are applied to groups of people to generate behavior change models. BCTs include techniques for eliciting changes in behavior of users. For example, a BCT can include rules related to communication with a user to elicit a behavior change, e.g. motivational rules. In various implementations, a taxonomy of BCTs can include a number of different BCTs in a hierarchical cluster. A large number of BCTs, e.g. 93, are gathered from a dynamic database of academic, medical, or other research institutions, to provide consistency across the number of health related applications and serving as a platform for the health related applications. In this way a user who is using a fitness/activity tracking app, a food logging app, and a medication reminder app, is not subjected to nudges that are contradictory in their behavior change models.

In a specific implementation, BCTs are applied to an experimental population to generate behavior change models for use in managing behavior changes of users. BCTs can be applied to an experimental population to see what changes in behavior are elicited through application of the BCTs. For example, BCTs can be applied to a population to see if target behaviors are elicited or if unwanted behaviors are elicited from the population. BCTs can be applied to an experimental population or subsets of an experimental population grouped based on one or a combination of behavior-specific behavior change phenotype variables, dynamic state responsiveness phenotype variables, and contexts associated with users. For example, BCTs can be applied to a subset of an experimental population including males between the ages of 40 and 50 living in a specific region. Through application of BCTs to an experimental population or subsets of the experimental population, behavior change models can be created. Behavior change models created through application of BCTs include population clusters grouped according to behavior-specific behavior change phenotype variables and behavior change outcomes exhibited through application of the BCTs to the experimental population. Behavior change models can include which BCTs were applied to a population leading, at least in part, to the grouping of the population or subsets of the population into the behavior change models.

In a specific implementation, a user is grouped into one or more behavior change models based, at least in part on their attributes, indicated by user data, potentially either or both activity data and context data, for the user. For example, a user can be grouped into a behavior change model for 40-year-old males with diabetes if attributes of the user indicate the user is a 40-year-old male with diabetes. After being grouped into one or more behavior change models, the behavior change models are applied to a user, e.g. BCTs of the behavior change models are applied to the user, and behavior outcomes are observed. Behavior change models can be updated or changed based on behavior outcomes observed after application of the behavior change models to users. For example, if application of a behavior change model to a user does not elicit a target behavior change, then the behavior change model can be updated. As part of updating a behavior change model, new BCTs can be added or old BCTs can be removed from the behavior change model. In another example, a behavior change model can be updated to target a different subset of a population according to behavior-specific behavior change phenotype variables.

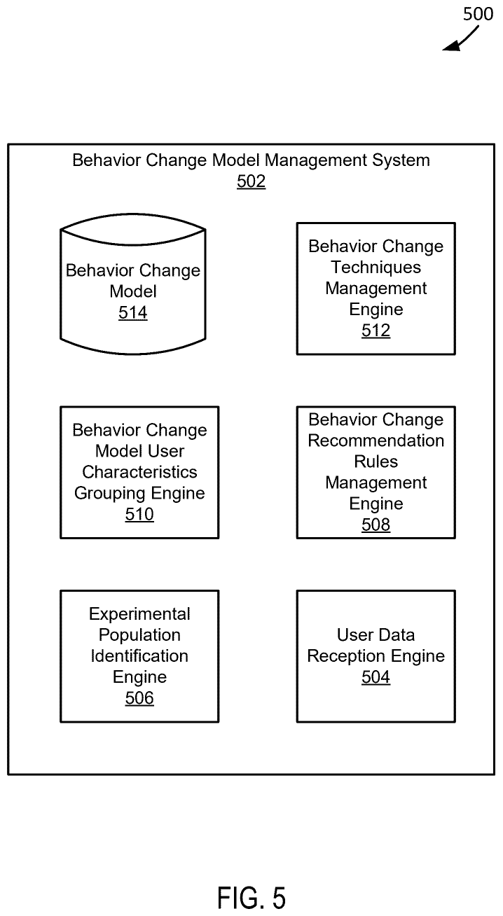

FIG. 5 depicts a diagram 500 of an example behavior change model management system 502. The behavior change model management system 502 is intended to represent a system that functions to maintain behavior change models for use in facilitating behavior changes in users. The behavior change model management system 502 can maintain behavior models based on user data, including activity data and context data, of a user. For example, if user data, potentially included in a user profile, indicates a user has successfully made a behavior change through application of a behavior change model, then the behavior change model management system 502 can update the behavior change model to indicate the successful results of behavior change in the user. In maintaining behavior change models, the behavior change model management system 502 can define or update one or a combination of behavior change module user characteristics, behavior change recommendation rules, and BCTs. For example, the behavior change model management system 502 can define specific behavior-specific behavior change phenotype variables and dynamic state responsiveness phenotype variables for a model, which are used in selecting the model for users based on their own phenotype variables. In another example, the behavior change model management system 502 can select motivational rules, included as part of BCTs to follow in applying a behavior model. The behavior change model management system 502 can be included as part of an applicable platform for facilitating behavior changes in users, such as the behavior change platforms described in this paper.

In a specific implementation, the behavior change model management system 502 functions to identify an experimental population or subset of an experimental population for use in maintaining behavior change models. For example, the behavior change model management system 502 can select users to include in an experimental population or a subset of an experimental population for use in maintaining behavior models to control facilitation of behavior changes in a user. The behavior change model management system 502 can identify an experimental population or subset of an experimental population based on phenotype variables. Specifically, the behavior change model management system 502 can select users based on either or both behavior-specific behavior change phenotype variables and dynamic state responsiveness phenotype variables to include in a behavior change model. For example, the behavior change model management system 502 can select males living in a specific region to include in a behavior model. In another example, the behavior change model management system 502 can select people suffering from the same disease in a specific demographic to include in a behavior model.

In a specific implementation, the behavior change model management system 502 functions to determine behavior change outcomes through application of a behavior change model. Behavior change outcomes determined by the behavior change model management system 502 can be used in maintaining behavior change models. For example, if users with a certain behavior-specific behavior change phenotype change their behaviors in a desired manner according to a behavior change model, then the behavior change model can be updated to include users with the phenotype in user characteristics associated with the model. The behavior change model management system 502 can utilize user data in determining behavior change outcomes. Specifically, the behavior change model management system 502 can utilize either or both user data extracted from data sent directly from a user and user data received from an application, e.g. a BCT application, to determine behavior change outcomes.

The example behavior change model management system 502 shown in FIG. 5 includes a user data reception engine 504, an experimental population identification engine 506, a behavior change recommendation rules management engine 508, a behavior change model user characteristics grouping engine 510, a behavior change techniques management engine 512, and a behavior change model datastore 514. The user data reception engine 504 is intended to represent an engine that functions to receive user data for use in managing behavior change models. Behavior change models managed by user data intercepted by the data reception engine 504 can be used to facilitate changes in user behaviors. User data received by the user data reception engine 504 can include either or both activity data and context data of a user.

In a specific implementation, the user data reception engine 504 functions to receive user data directly from a user. Specifically, the user data reception engine 504 can receive user data including activity data directly from a user. For example, the user data reception engine 504 can receive activity data indicating a number of steps a user has taken in a day directly from an applicable device for tracking activity of a user, such as the activity and context monitoring devices described in this paper. Additionally, the user data reception engine 504 can receive context data indicating a context associated with a user directly from the user. For example, the user data reception engine 504 can receive context data indicating a current time of day for a user directly from the user. The user data reception engine 504 can extract user data from data as it is sent from a user to an applicable destination, such as a BCT application.

In a specific implementation, the user data reception engine 504 functions to receive user data from a third party source. For example, the user data reception engine 504 can receive user data from one or a plurality of BCT applications. Specifically, the user data reception engine 504 can receive either or both activity data and context data from a third party source, such as a calendar application or a BCT application. The user data reception engine 504 can receive user data generated or otherwise extracted at a third party source from the third party source. For example, the user data reception engine 504 can receive activity data indicating a medical condition of a user from a hospital record application that generates the activity data.

The experimental population identification engine 506 is intended to represent an engine that functions to identify an experimental population of people together for use in maintaining a behavior change model. In identifying an experimental population of people, the experimental population identification engine 506 can group together people, either real or simulated, to create an experimental population or a subset of an experimental population based on characteristics of the people for use in maintaining a behavior change model. More specifically, the experimental population identification engine 506 can group people together to form an experimental population or subset thereof, and a behavior change model can be applied to the population or subset to determine if behavior changes are actually achieved in the population or subset. The experimental population identification engine 506 can group together an experimental population based on one or a combination of behavior-specific behavior change phenotype variables, dynamic state responsiveness phenotype variables, and contexts associated with people. For example, the experimental population identification engine 506 can group together people who live in the same region and suffer from the same disease. In identifying an experimental population, the experimental population identification engine 506 can add and remove users to and from an already identified experimental population.

In a specific implementation, the experimental population identification engine 506 functions to identify an experimental population based on input received from an applicable source. For example, a health care provider can identify users that should be grouped together in an experimental population based on phenotype variables, and the experimental population identification engine 506 can subsequently group the users together to form the experimental population. In another example, a life coach can identify users that should be grouped together in an experimental population based on phenotype variables, and the experimental population identification engine 506 can subsequently group the users together to form the experimental population.

In a specific implementation, the experimental population identification engine 506 functions to identify an experimental population based on behavior change outcomes. For example, the experimental population identification engine 506 can identify an experimental population based on success in achieving desired behavior changes or lack thereof as a result of application of a behavior change model. More specifically, the experimental population identification engine 506 can identify an experimental population based on behavior change outcomes of the users in the population through application of a specific behavior change model. In identifying an experimental population based on behavior change outcomes, the experimental population identification engine 506 can add and remove users to and from an experimental population, e.g. on a behavior-specific behavior change phenotype basis. For example, if users with a specific behavior change phenotype are seeing desired behavior changes through application of a specific behavior change model, then the experimental population identification engine 506 can add additional users to an experimental population. Alternatively, in another example, if users with a specific behavior-specific behavior change phenotype in an experimental population are not experiencing desired behavior changes through application of a behavior change model, then the experimental population identification engine 506 can remove the users from the experimental population, e.g. on a behavior-specific behavior change phenotype basis. Further in the example, the behavior change model can be reapplied to the modified experimental population to determine new behavior change outcomes, e.g. whether desired changes are observed.

The behavior change recommendation rules management engine 508 is intended to represent an engine that functions to maintain behavior change recommendation rules for a behavior change model. In maintaining behavior change recommendation rules for a behavior change model, the behavior change recommendation rules management engine 508 can add or delete behavior change recommendation rules from a behavior change model and edit behavior change recommendation rules in a behavior change model. For example, the behavior change recommendation rules management engine 508 can add behavior recommendation rules to a behavior change model as part of generating the behavior change model. In another example, if behavior change recommendation rules specify a person with a certain behavior-specific behavior change phenotype should change their diet, then the behavior change recommendation rules management engine 508 can modify the rules to include the person should change both their diet and their fitness activity level.

In a specific implementation, the behavior change recommendation rules management engine 508 functions to maintain behavior change recommendation rules based on received input. The behavior change recommendation rules management engine 508 can maintain behavior change recommendation rules based on input received from an applicable source or authority. For example, a health care provider can provide specific behaviors that users with a certain behavior-specific behavior change phenotype should change and the behavior change recommendation rules management engine 508 can subsequently create behavior change recommendation rules indicating users with the certain phenotype should change the specific behaviors. Additionally, the behavior change recommendation rules management engine 508 can generate input by querying an applicable data source, e.g. from a dynamic database of academic, medical, or other research institutions. For example, the behavior change recommendation rules management engine 508 can query a cancer society database to generate input indicating desired changes for patients with a specific type of cancer.

In a specific implementation, the behavior change recommendation rules management engine 508 functions to maintain behavior change recommendation rules based on behavior change outcomes observed through application of a behavior change model. For example, if a desired behavior change of running ten miles a week is not being attained through application of a behavior change model, then the behavior change recommendation rules management engine 508 can change the behavior change recommendation rules in the behavior change model to indicate running five miles a week as the desired change. The behavior change recommendation rules management engine 508 can maintain behavior change recommendation rules based on application of a behavior change model to an experimental population or subset of a population identified by an applicable engine, such as the experimental population identification engines described in this paper. More specifically, the behavior change recommendation rules management engine 508 can use behavior change outcomes observed through application of a behavior change model to an experimental population, potentially multiple times, to maintain behavior change recommendation rules in the behavior change model.

The behavior change model user characteristics grouping engine 510 is intended to represent an engine that functions to define user characteristics of a behavior change model. User characteristics of a behavior change model include characteristics of users, e.g. behavior-specific behavior change phenotypes, used to match the user to the behavior change model for purposes of facilitating behavior changes of the users. User characteristics of a behavior change model can include one or a combination of values of behavior-specific behavior change phenotype variables, values of dynamic state responsiveness phenotype variables, and contexts associated with or capable of being associated with a user. For example, user characteristics of a behavior change model can include users who are between 30 and 40 years old with a goal of completing a marathon. Further in the example, the behavior change model can include a behavior change recommendation rule indicating to run ten miles every week for the next ten weeks. In defining user characteristics of a behavior change model, the behavior change model user characteristics grouping engine 510 can modify user characteristics already defined for a behavior change model. For example, if user characteristics defined for a behavior change model include people between the ages of 20 and 50, then the behavior change model user characteristics grouping engine 510 can modify the user characteristic to only include people between the ages of 20 and 30.

In a specific implementation, the behavior change model user characteristics grouping engine 510 functions to define user characteristics of a behavior change model based on received input. The behavior change model user characteristics grouping engine 510 can define user characteristics of a behavior change model based on input received from an applicable source or authority. For example, a health care provide can provide certain behavior-specific behavior change phenotypes to define for a behavior change module and the behavior change model user characteristics grouping engine 510 can subsequently define user characteristics of the behavior change module to include phenotype variables that define the specific behavior change module. Additionally, the behavior change model user characteristics grouping engine 510 can generate input by querying an applicable data source, e.g. from a dynamic database of academic, medical, or other research institutions. For example, the behavior change model user characteristics grouping engine 510 can query an academic society database to generate input indicating user characteristics for a behavior change model for eliciting changes in behaviors of users diagnosed with a specific disease.

In a specific implementation, the behavior change model user characteristics grouping engine 510 functions to identify user characteristics for a behavior change model based on behavior change outcomes observed through application of the behavior change model. For example, if a desired behavior change of losing five pounds in a week is not being attained in people with a specific phenotype through application of a behavior change model, then the behavior change model user characteristics grouping engine 510 can change user characteristics defined for the model to exclude people with the specific phenotype. The behavior change model user characteristics grouping engine 510 can define user characteristics for a behavior change model based on application of a behavior change model to an experimental population or subset of a population identified by an applicable engine, such as the experimental population identification engines described in this paper. More specifically, the behavior change model user characteristics grouping engine 510 can use behavior change outcomes observed through application of a behavior change model to an experimental population, potentially multiple times, to define user characteristics for the behavior change model.

The behavior change techniques management engine 512 is intended to represent an engine that functions to maintain BCTs for a behavior change model. BCTs of a behavior change model, maintained by the behavior change techniques management engine 512 can be used to control communications for purposes of facilitating behavior changes in a user. For example, the behavior change techniques management engine 512 can set motivational rules as part of BCTs specifying to send contextual notifications to a user based on a context associated with the user. In another example, the behavior change techniques management engine 512 can set rules for controlling sending of dynamic escalation nudges to people affiliated with a user. In maintaining BCTs for a behavior change model, the behavior change techniques management engine 512 can generate and update the BCTs of a behavior change model. For example, if a BCT is not working through application of a behavior change model, then the behavior change techniques management engine 512 can remove or otherwise dissociate the BCT from the behavior change model.

In a specific implementation, the behavior change techniques management engine 512 functions to associate or otherwise include user contexts as part of BCTs of a behavior change model. User context includes contexts associated with a user. In including user contexts as part of BCTs, the behavior change techniques management engine 512 can associate contexts a user is capable of being at with rules included as part of the BCTs for use in facilitating behavior changes in users based on context. In associating contexts with rules, the behavior change techniques management engine 512 can make the rules dependent on context. For example, the behavior change techniques management engine 512 can cause a specific rule to be selected for application when a user has a specific context. In another example, the behavior change techniques management engine 512 can define a rule to be followed according to a specific user context.

In a specific implementation, the behavior change techniques management engine 512 functions to maintain a taxonomy of BCTs for a behavior change model. In maintaining a taxonomy of BCTs, the behavior change techniques management engine 512 can group together a plurality of BCTs to form, at least in part, the taxonomy of BCTs. For example, the behavior change techniques management engine 512 can group together techniques for changing behaviors in people who suffer from hypertension. Further, in maintaining a taxonomy of BCTs, the behavior change techniques management engine 512 can arrange BCTs grouped together into an ordered hierarchy based on one or a plurality of applicable factors for organizing the BCTs into an ordered hierarchy. For example, the behavior change techniques management engine 512 can arrange BCTs for training to run a marathon into a hierarchy based on a stage in training in which each BCT is applied.

In a specific implementation, the behavior change techniques management engine 512 functions to manage BCTs of a behavior change model based on received input. The behavior change techniques management engine 512 can manage BCTs of a behavior change model based on input received from an applicable source or authority. For example, a dietitian can provide certain diet recommendations for improving the health of a user who has Celiac disease and the behavior change techniques management engine 512 can subsequently include rules for facilitating users to cat according to the diet recommendations as part of BCTs of a behavior change module. Additionally, the behavior change techniques management engine 512 can generate input by querying an applicable data source, e.g. from a dynamic database of academic, medical, or other research institutions. For example, the behavior change techniques management engine 512 can query an academic society database to generate input indicating motivational rules to follow in facilitating a user with a specific disease to change their behaviors for purposes of curing the disease.

In a specific implementation, the behavior change techniques management engine 512 functions to maintain BCTs for a behavior change model based on behavior change outcomes observed through application of the behavior change model. For example, if a desired behavior change of ability to complete a triathlon is not being attained in people with a specific phenotype through application of a behavior change model, then the behavior change techniques management engine 512 can change BCTs for the model to target users with the specific phenotype. The behavior change techniques management engine 512 can maintain BCTs for a behavior change model based on application of a behavior change model to an experimental population or subset of a population identified by an applicable engine, such as the experimental population identification engines described in this paper. More specifically, the behavior change techniques management engine 512 can use behavior change outcomes observed through application of BCTs of a behavior change model to an experimental population, potentially multiple times, to maintain BCTs for a behavior change model.

The behavior change model datastore 514 is intended to represent a datastore that functions to store behavior change model data indicating behavior change models for use in application in facilitating behavior changes in users. The behavior change model datastore 514 can store behavior change model data to include behavior change recommendation rules, behavior change model user characteristics, and BCTs for a behavior change model. Behavior change model data stored in the behavior change model datastore 514 can be maintained, at least in part, by an applicable engine for maintaining behavior change recommendation rules, such as the behavior change recommendation rules management engines described in this paper. Further, behavior change model data stored in the behavior change model datastore 514 can be maintained, at least in part, by an applicable engine for maintaining user characteristics of a behavior change model, such as the behavior change model user characteristics grouping engines described in this paper. Additionally, behavior change model data stored in the behavior change model datastore 514 can be maintained, at least in part, by an applicable engine for maintaining BCTs of a behavior change model, such as the behavior change techniques management engines described in this paper.

In an example of operation of the example behavior change model management system 502 shown in FIG. 5, the user data reception engine 504 functions to receive user activity data of a user in facilitating behavior changes of the user in response to application of a behavior change model. In the example of operation of the example system shown in FIG. 5, the user is part of an experimental population identified by the experimental population identification engine 506 and the user activity data is used to determine behavior change outcomes of the experimental population based on application of the behavior change model to the experimental population. Further, in the example of operation of the example system shown in FIG. 5, the behavior change recommendation rules management engine 508 maintains behavior change rules for the behavior change model. In the example of operation of the example system shown in FIG. 5, the behavior change model user characteristics grouping engine 510 defines user characteristics for application of the behavior change model based on the determined behavior change outcomes. Additionally, in the example of operation of the example system shown in FIG. 5, the behavior change techniques management engine 512 maintains BCTs of the behavior change model based on the determined behavior change outcomes.

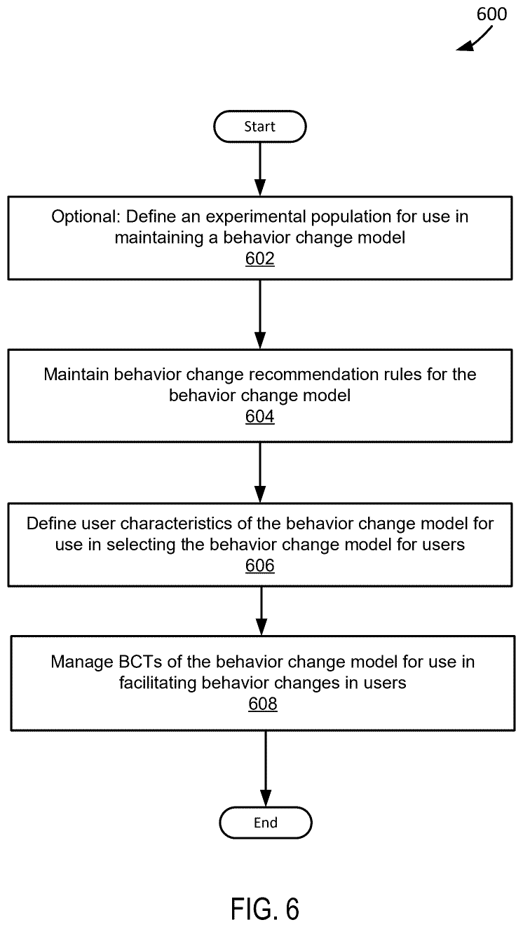

FIG. 6 depicts a flowchart 600 of an example of a method for maintaining a behavior change model for use in facilitating behavior changes in users. The flowchart 600 optionally begins at module 602, where an experimental population for use in maintaining a behavior change model is defined. An applicable engine for defining an experimental population for use in maintaining a behavior change model, such as the experimental population identification engines described in this paper. A defined experimental population can be used in maintaining a behavior change model by observing behavior change outcomes that occur in response to application, potentially multiple times, of the behavior change model to the experimental population. An experimental population can be defined according to input. For example, an experimental population can be defined according to input received from a health care authority or generated by querying an academic database. Additionally, an experimental population can be defined based on behavior change outcomes observed through application of a behavior change model to the experimental population. For example, users in the experimental population can be removed from the experimental population if the users are failing to change at all through application of a behavior change model to the users.

The flowchart 600 continues to module 604, where behavior change recommendation rules for the behavior change model are maintained. An applicable engine for maintaining behavior change recommendation rules for a behavior change model, such as the behavior change recommendation rules management engines described in this paper, can maintain behavior change recommendation rules for the behavior change model. Behavior change recommendation rules can be maintained for the behavior change model based on input. For example, behavior change recommendation rules can be maintained based on input generated by querying a research institution database. Additionally, behavior change recommendation rules can be maintained for the behavior change model based on behavior change outcomes observed through application of a behavior change model to the experimental population.

The flowchart 600 continues to module 606, where user characteristics of the behavior change model for use in selecting the behavior change model for users are defined. An applicable engine for defining user characteristics for a behavior change model, such as the behavior change model user characteristics grouping engines described in this paper, can define user characteristics of the behavior change model for use in selecting the behavior change model. User characteristics defined for the behavior change model can include one or a plurality of behavior-specific behavior change phenotypes for use in selecting the behavior change mode for users with the one or a plurality of behavior-specific behavior change phenotypes. User characteristics can be defined for the behavior change model based on input. For example, a health care provider can define a behavior-specific behavior change phenotype for the behavior model, and user characteristics of the behavior change model can be defined to include the behavior-specific behavior change phenotype. Additionally, user characteristics can be defined for the behavior change model based on behavior change outcomes observed through application of a behavior change model to the experimental population. For example, if members in the experimental population with a specific phenotype are failing to change in response to application of the behavior change model, then the specific phenotype can be removed from user characteristics of the behavior change model.

The flowchart 600 continues to module 608, where BCTs of the behavior change model are managed. An applicable engine for managing BCTs of a behavior change model, such as the behavior change techniques management engines described in this paper, can manage BCTs of the behavior change model. In managing BCTs of the behavior change model BCTs can be associated with the behavior change model, and BCTs associated with the behavior change model can be modified or dissociated from the behavior change model. BCTs of the behavior change model can be associated with user contexts, for use in facilitating behavior changes in users based on user contexts. BCTs of the behavior change model can be maintained according to input. Additionally, BCTs of the behavior change model can be maintained according to behavior change outcomes observed through application of the behavior change model to the experimental population. For example, if behavior change outcomes indicate a particular BCT is failing to produce desired behavior changes in the experimental population, then the particular BCT can be modified or dissociated from the behavior change model.

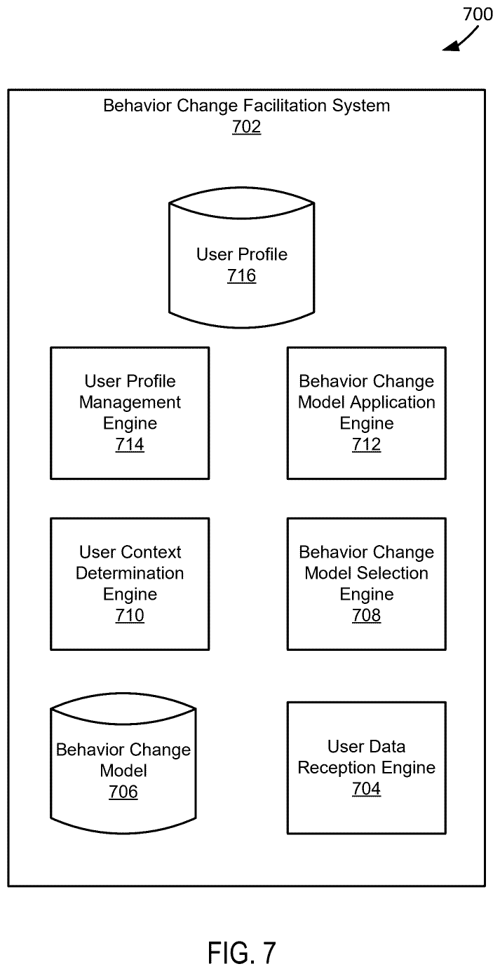

FIG. 7 depicts a diagram 700 of an example behavior change facilitation system 702. The behavior change facilitation system 702 is intended to represent a system that functions to apply a behavior change model to a user for facilitating behavior changes in the user. In applying a behavior change model to a user, the behavior change facilitation system 702 can match a user to a specific behavior change model based on a behavior-specific behavior change phenotype of the user. Further, in applying a behavior change model to a user, the behavior change facilitation system 702 can determine a context associated with the user for use in applying the behavior change model to the user. Additionally, the behavior change facilitation system 702 can apply a behavior change model to a user based on a determined context associated with the user. The behavior change facilitation system 702 can be included as part of an applicable platform for facilitating behavior changes in users, such as the behavior change platforms described in this paper.

In a specific implementation, the behavior change facilitation system 702 functions to generate behavior change communication instructions for use in controlling communication with the user or a person associated with the user as part of facilitating behavior changes in the user. Behavior change communication instructions include applicable instructions for controlling communication with a user or a person associated with the user for purposes of facilitating behavior change in the user. For example, behavior change communication instructions can specify content to produce to a user or person associated with the user, a manner in which to produce the content for the user or the person, and a time at which to produce the content for the user or the person. The behavior change facilitation system 702 can generate behavior change communication instructions as part of applying a behavior change model to a user. For example, if motivational rules of a behavior change model specify to remind a user of their goal of completing a marathon, then the behavior change facilitation system 702 can generate behavior change communication instructions specifying to send a message to the user reminding them of their goal. The behavior change facilitation system 702 can generate behavior change communication instructions used to send a specific message to a user based on a context associated with the user. For example, if BCTs specify to send a dynamic escalation nudge to a doctor if a user fails to take their medication, and the user has failed to take their medication, then the behavior change facilitation system 702 can generate behavior change communication instructions to control sending of the nudge to the doctor.

The behavior change facilitation system 702 shown in FIG. 7 includes a user data reception engine 704, a behavior change model datastore 706, a behavior change model selection engine 708, a user context determination engine 710, a behavior change model application engine 712, a user profile management engine 714, and a user profile datastore 716. The user data reception engine 704 is intended to represent an applicable engine that functions to receive user data for purposes of facilitating behavior changes in users, such as the user data reception engines described in this paper. The user data reception engine 704 can receive user data including either or both activity data of a user and context data of a user. The user data reception engine 704 can receive user data from an applicable source. For example, the user data reception engine 704 can receive user data directly from a user. In another example, the user data reception engine 704 can extract user data from a stream of data sent from a user to an applicable destination, e.g. a BCT application. In yet another example, the user data reception engine 704 can receive user data from a BCT application.

The behavior change model datastore 706 is intended to represent an applicable datastore for storing behavior change model data indicating behavior change models, such as the behavior change model datastores described in this paper. Behavior change model data stored in the behavior change model datastore 706 can be maintained by an applicable system for maintaining behavior change models, such as the behavior change model management systems described in this paper. Behavior change model data stored in the behavior change model datastore 706 can include defined user characteristics of a behavior change model for use in selecting the model for users based on the user characteristics. For example, behavior change model data stored in the behavior change model datastore 706 can include behavior-specific behavior change phenotypes defined for a behavior change model that are used to map or otherwise select the model for users based on their phenotypes. Additionally, behavior change model data stored in the behavior change model datastore 707 can include BCTs, including context-specific BCTs to follow in facilitating behavior change through application of a behavior change model.

The behavior change model selection engine 708 is intended to represent an engine that functions to select a behavior change model for a user for purposes of facilitating behavior change in the user. The behavior change model selection engine 708 can select a behavior change model based on received user data. For example, if user data indicates a user wishes to climb Mount Everest, then the behavior change model selection engine 708 can select a behavior change model to change behaviors of people in training for high altitude mountain climbing. Additionally, the behavior change model selection engine 708 can select a behavior change model based on one or a combination of a values of behavior-specific behavior change phenotype variable values of a user, dynamic state responsiveness phenotype variable values of a user, and contexts of a user. For example, if a behavior-specific behavior change phenotype matches, at least in part, user characteristics defined for a behavior change model, then the behavior change model selection engine 708 can select for the user, or otherwise map the user to, the behavior change model. The behavior change model selection engine 708 can select a behavior change model for a user based on behavior change model data stored in an applicable datastore, such as the behavior change model datastores described in this paper.

In a specific implementation, the behavior change model selection engine 708 functions to select a behavior change model for a user based on a user profile maintained for a user. In selecting a behavior change model for a user based on a user profile of a user, the behavior change model selection engine 708 can select the model based on values of dynamic state responsiveness phenotype variables of the user. For example, if a user profile indicates a user is currently depressed, then the behavior change model selection engine 708 can select a behavior change model based on the user's current state of being depressed. In another example, if a user profile indicates a user has achieved a desired behavior change of a behavior change model applied to the user, then the behavior change model selection engine 708 can select another behavior change model to facilitate another behavior change in the user.

In a specific implementation, the behavior change model selection engine 708 functions to select a behavior change model for a user based on behavior change outcomes observed through application of the behavior change model. Additionally, the behavior change model selection engine 708 can select a behavior change model for a user based on behavior change outcomes observed through application of a previous or currently selected behavior change model to the user. For example, if desired behavior change outcomes are not seen in a user through application of a first behavior change model to the user, then the behavior change model selection engine 708 can select a new behavior change model for application to the user to achieved the behavior change outcomes.

The user context determination engine 710 is intended to represent an engine that functions to determine a context associated with a user at a specific time. The user context determination engine 710 can determine either or both a current or future context associated with a user. For example, the user context determination engine 710 can determine a future location a user will occupy by accessing a calendar of the user. The user context determination engine 710 can determine a context associated with a user based on context data included as part of user data and received from an applicable device, such as the activity and context monitoring devices described in this paper. For example, a step tracking wearable device of a user can provide a current location of the user, as part of context data, to the user context determination engine 710 which can subsequently identify a current context associated with the user as occupying the current location. In another example, the user context determination engine 710 can access an email account of a user to determine the user is currently in a happy mood.

The behavior change model application engine 712 is intended to represent an engine that functions to apply a behavior change model for purposes of facilitating behavior changes in a user. In applying a behavior change model, the behavior change model application engine 712 can apply BCTs of a behavior change model in facilitating behavior changes in the user. Further, in applying a behavior change model, the behavior change model application engine 712 can apply BCTs of a behavior change model according to received user data. For example, if activity data indicates a user is currently running, and a motivational rule of an applied behavior change model indicates encouraging a user while they are exercising, then the behavior change model application engine 712 can facilitate encouraging the user while they are running.

In a specific implementation, the behavior change model application engine 712 functions to apply a behavior change model based on a context associated with a user. In applying a behavior change model according to a context associated with a user, the behavior change model application engine 712 select BCTs to apply based on the context. For example, if a behavior change model includes motivational rules to specifically apply in the morning, and context indicates it is currently the morning at the location of the user, then the behavior change model application engine 712 can apply the motivational rules. Additionally, in applying a behavior change model according to a context associated with a user, the behavior change model application engine 712 can apply BCTs based on the specific context. For example, if a BCTs specifies presenting a user's current heart rate, as indicated by a context associated with the user, then the behavior change model application engine 712 can facilitate presentation of content to the user including the user's current heart rate.

In a specific implementation, the behavior change model application engine 712 functions to generate behavior change communication instructions through application of a behavior change model. More specifically, the behavior change model application engine 712 can generate behavior change communication instructions according to BCTs of a behavior change model. For example, if a BCT instructs to produce a soothing message to a person who suffers from anxiety, then the behavior change model application engine 712 can generate communication instructions for use in facilitating production of the soothing message to the person. The behavior change model application engine 712 can generate behavior change communication instructions according to BCTs of a behavior change model and a context associated with a user.

In a specific implementation, the behavior change model application engine 712 functions to apply a behavior change model based on a user profile. For example, if a user profile indicates a specific behavior change model has been selected for facilitating behavior changes in the user, then the behavior change model application engine 712 can apply the specific behavior change model according to the user profile. The behavior change model application engine 712 can apply a behavior change model based on behavior change outcomes observed through application of the model, as indicated by a user profile. For example, if a user profile indicates a person has achieved running a mile in under seven minutes through application of a behavior change model, then the behavior change model application engine 712 can apply BCTs in the behavior change model to facilitate the user running a mile in under six minutes.

In a specific implementation, the behavior change model application engine 712 functions to apply a taxonomy of BCTs in applying a behavior change model for purposes of facilitating behavior change in a user. In applying a taxonomy of BCTs, the behavior change model application engine 712 can select a BCT in the taxonomy to apply and subsequently apply the BCT. For example, the behavior change model application engine 712 can select a first level BCT to apply from a BCT taxonomy and select a second level BCT to apply from the taxonomy based on selection and application of the first level BCT. In applying a taxonomy of BCTs, the behavior change model application engine 712 can apply the taxonomy according to a user context. For example, the behavior change model application engine 712 can select a BCT in a taxonomy of BCTs to apply based on a user context and subsequently apply the BCT based on the user context.

The user profile management engine 714 is intended to represent an engine that functions to maintain a user profile of a user for purposes of facilitating behavior change in the user. The user profile management engine 714 can maintain a user profile based on received user data. For example, the user profile management engine 714 can update a user profile to indicate observed behavior changes in a user, as indicated by received user data. Additionally, the user profile management engine 714 can maintain a user profile based on a behavior change model selected for a user. For example, the user profile management engine 714 can update a user profile to indicate one or a plurality of behavior change models selected for a user. Further, the user profile management engine 714 can maintain a user profile based on determined user contexts. For example, the user profile management engine 714 can update a user profile to indicate a determined user context and a specific time at which the user has or will have the context.

The user profile datastore 716 functions to store user profile data of a user profile maintained as part of facilitating behavior change in the user. User profile data stored in the user profile datastore 716 can indicate one or a combination of observed behavior changes in a user, contexts of a user, a behavior change model selected for a user, and data related to application of the behavior change model. User profile data stored in the user profile datastore 716 can be maintained by an applicable engine for maintaining a user profile, such as the user profile management engines described in this paper.

In an example of operation of the example behavior change facilitation system 702 shown in FIG. 7, the user data reception engine 704 receives user data indicating a behavior-specific behavior change phenotype of a user. In the example of operation of the example system shown in FIG. 7, the behavior change model datastore 706 stores behavior change model data indicating behavior change models for use in facilitating behavior changes in users. Further, in the example of operation of the example system shown in FIG. 7, the behavior change model selection engine uses the behavior change model data stored in the behavior change model datastore 706 and the phenotype of the user indicated by the user data to select a behavior change model for use in facilitating behavior change in the user. In the example of operation of the example system shown in FIG. 7, the user context determination engine 710 determines a user context from the user data. Additionally, in the example of operation of the example system shown in FIG. 7, the behavior change model application engine 712 applies the behavior change model according to the user context for facilitating the behavior changes in the user. In the example of operation of the example system shown in FIG. 7, the user profile management engine 714 maintains a user profile of the user based on application of the behavior change model according to the user context.

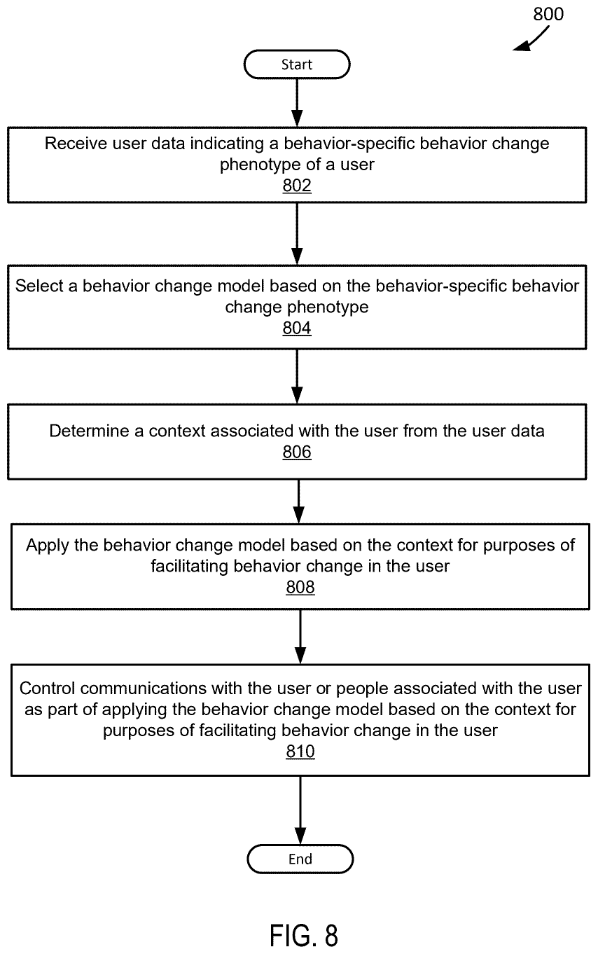

FIG. 8 depicts a flowchart 800 of an example of a method of applying a behavior change model for purposes of facilitating behavior change in a user. The flowchart 800 begins at module 802, where user data indicating a behavior-specific behavior change phenotype of a user is received. An applicable engine for receiving user data, such as the user data reception engines described in this paper, can receive user data indicating a behavior-specific behavior change phenotype of a user. User data indicating a behavior-specific behavior change phenotype of a user can be received directly from a user or from an applicable source, such as a BCT application. For example, user data indicating a behavior-specific behavior change phenotype of a user can be received directly from one or a plurality of context monitoring devices. Additionally, user data indicating a behavior-specific behavior change phenotype of a user can be extracted from an intercepted data stream between a user and an applicable source or destination, such as a BCT application.

The flowchart 800 continues to module 804, where a behavior change model is selected based on the behavior-specific behavior change phenotype of the user. An applicable engine for selecting a behavior change model to apply in facilitating behavior changes in a user, such as the behavior change model selection engines described in this paper, can select a behavior change model based on the behavior-specific behavior change phenotype of the user. For example, a behavior change model can be selected by matching one or a plurality of values of behavior-specific behavior change phenotype variables and dynamic state responsiveness phenotype variables of the user to user characteristics defined for a behavior change model.

The flowchart 800 continues to module 806, where a context associated with the user is determined from the user data. An applicable engine for determining context associated with users from user data, such as the user context determination engines described in this paper, can determine a context associated with the user from the user data. For example, a current location of the user can be determined from the user data.

The flowchart 800 continues to module 808, where the behavior change model is applied to the user based on the context for purposes of facilitating behavior change in the user. An applicable engine for applying a behavior change model based on context associated with a user, such as the behavior change model application engines described in this paper, can apply the behavior change model to the user based on the context. In applying the behavior change model based on the context, BCTs of the behavior change model can be selected based on the context. Additionally, in applying the behavior change model based on the context, BCTs of the behavior change model can be applied, or otherwise followed, based on the context.

The flowchart 800 continues to module 810, where communications with the user or people associated with the user are controlled as part of applying the behavior change model based on the context for purposes of facilitating behavior change in the user. In controlling communication with the user or people associated with the user, behavior change communication instructions can be generated to control communications according to application of the behavior change model. For example, BCTs of the behavior change model can be applied according to the context to generate user behavior change communications instructions for use in controlling communications with the user or people associated with the user. Further in the example, either or both contextual notifications and dynamic escalation nudges can be controlled based on application of the BCTs of the behavior change model applied according to the context.

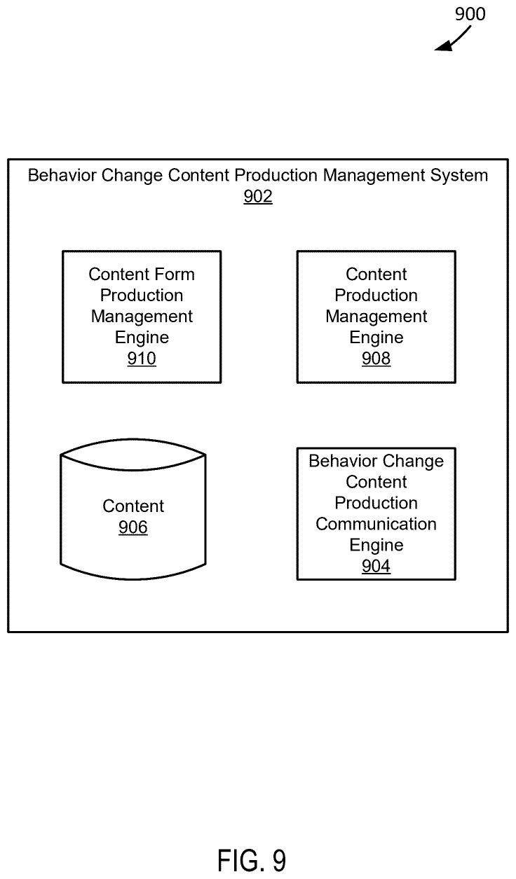

FIG. 9 depicts a diagram 900 of a behavior change content production management system 902. The behavior change content production management system 902 is intended to represent a system that functions to control communications with a user or people associated with the user to facilitate behavior changes in the user. The behavior change content production management system 902 can be included as part of an applicable platform for facilitating behavior changes in users, such as the behavior change platforms described in this paper.

In a specific implementation, in controlling communications with a user or people associated with the user, the behavior change content production management system 902 functions to either or both select and generate content to produce for either or both the user or the people. For example, the behavior change content production management system 902 can generate content data used to produce a textual message indicating a fitness goal of a user. Further, in controlling communications with a user or people associated with the user, the behavior change content production management system 902 can select a form in which to produce content. For example, the behavior change content production management system 902 can generate content production instructions, included as part of content data, instructing to produce content as an auditory message. The behavior change content production management system 902 can provide content data, including content production instructions, to an applicable device for producing content, such as the behavior change nudge devices described in this paper, which can subsequently produce the content using the content data.

In a specific implementation, the behavior change content production management system 902 functions to control communications with a user or people associated with the user based on application of a behavior change model to the user. Specifically, the behavior change content production management system 902 can control communication with a user or people associated with a user based on behavior change communication instructions generated through application of a behavior change model to the user. For example, if behavior change communication instructions indicate displaying a motivating image to a user, then the behavior change content production management system 902 can generate content data including data used to produce a motivating image. Further in the example, the behavior change content production management system 902 can provide the generated content data to a behavior change nudge device, which can subsequently produce the motivating image using the content data.

The behavior change content production management system 902 shown in FIG. 9 includes a behavior change content production communication engine 904, a content datastore 906, a content production management engine 908, and a content form production management engine 910. The behavior change content production communication engine 904 is intended to represent an engine that functions to send and receive data for purposes of facilitating behavior change in a user. The behavior change content production communication engine 904 can send content data to an applicable device for producing content for purposes of facilitating behavior change in a user, such as the behavior change nudge devices described in this paper. For example, the behavior change content production communication engine 904 can send content data used to produce a contextual nudge for a user. The behavior change content production communication engine 904 can send data in response to behavior change communication instructions. For example, if behavior change communication instructions indicate to send a dynamic escalation to a person associated with a user, then the behavior change content production communication engine 904 can send content data used to produce the dynamic escalation to the person's device.

In a specific implementation, the behavior change content production communication engine 904 functions to gather or receive content data for use in producing content to facilitate behavior change in a user. The behavior change content production communication engine 904 can gather or receive content data from an applicable source. For example, the behavior change content production communication engine 904 can receive content data from a user or an application utilized by a user, e.g. a BCT application. In another example, the behavior change content production communication engine 904 can gather content data from a dynamic database of academic, medical, or other research institutions.

The content datastore 906 is intended to represent a datastore that functions to store content data. Content data stored in the content datastore 906 can be used to produce content to a user or a person associated with a user for purposes of facilitating behavior change in the user. The content datastore 906 can store content data gathered or received from an applicable source. Additionally, the content datastore 906 can store content data generated at the behavior change content production management system 902 in response to behavior change communication instructions. For example, content data stored in the content datastore 906 can include content data generated to produce a specific message according to behavior change communication instructions.

The content production management engine 908 is intended to represent an engine that functions to manage provisioning of content data for use in producing content to facilitate behavior changes in a user. The content production management engine 908 can instruct an applicable engine for communicating for purposes of producing content to facilitate behavior changes, such as the behavior change content production communication engines described in this paper, to provide content data. The content production management engine 908 can manage provisioning of content according to behavior change communication instructions. For example, if behavior change communication instructions indicate to send a reminder to a patient to take their medicine as part of facilitating behavior change, then the content production management engine 908 can cause content data for producing the reminder to be sent to the patient's device.

In a specific implementation, in managing provisioning of content data, the content production management engine 908 functions to generate or receive content data. The content production management engine 908 can gather content data from an applicable source. For example, the content production management engine 908 can gather content data from a dynamic database of an academic institution. Additionally, the content production management engine 908 can generate or receive content data according to behavior change communication instructions. For example, the content production management engine 908 can generate content data used to produce a specific message as indicated by behavior change communication instructions.

The content form production management engine 910 is intended to represent an engine that functions to manage a form in which content is produced for purposes of facilitating behavior change in a user. In managing a form in which content is produced, the content form production management engine 910 can generate content production instructions, as included as part of content data, indicating a form in which to produce content. The content form production management engine 910 can manage a form for producing content based on behavior change communication instructions. Specifically, the content form production management engine 910 can generate content production instructions specifying to produce content in a certain form, as indicating by behavior change communication instructions. Additionally, the content form production management engine 910 can manage a form for producing content based on a user profile of a user. For example, if a user profile specifies a user prefers receiving messages through a specific e-mail service, then the content form production management engine 910 can generate content production instructions specifying to send messages to the user through the specific e-mail service.

In an example of operation of the example behavior change content production management system 902 shown in FIG. 9, the content production management engine 908 generates content data used in producing content for facilitating behavior changes in a user based on behavior change communication instructions. In the example of operation of the example system shown in FIG. 9, the content form production management engine 910 generates content production instructions, included as part of the content data, indicating a form in which to produce the content. Further in the example of operation of the example system shown in FIG. 9, the behavior change content production communication engine 904 provides the content data to an applicable device for purposes of producing the content to facilitate the behavior changes in the user.

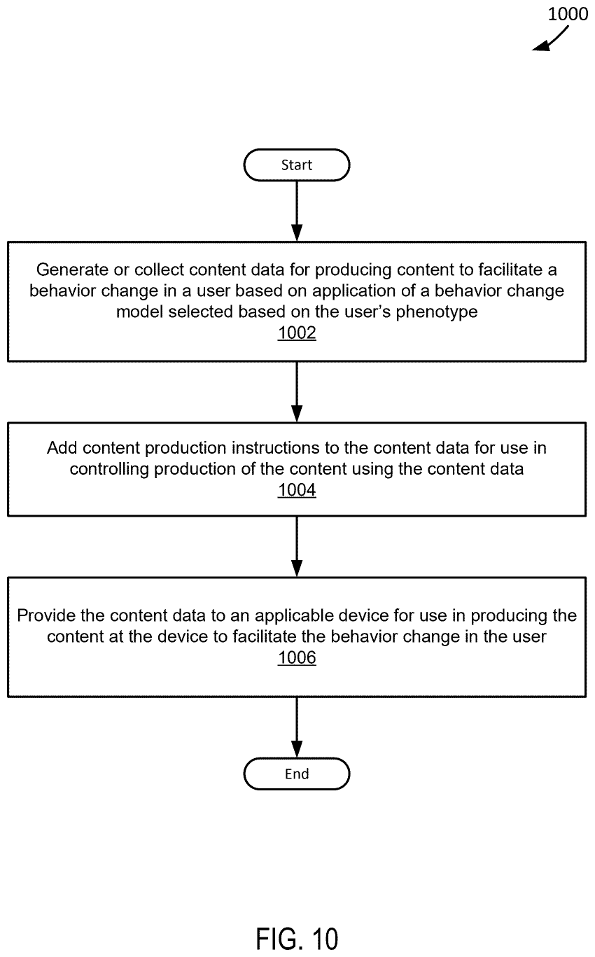

FIG. 10 depicts a flowchart 1000 of an example of a method for controlling production of content to facilitate behavior changes in a user through application of a behavior change model selected based on the user's phenotype. The flowchart 1000 begins at module 1002 where content data for producing content to facilitate a behavior change in a user through application of a behavior change model selected based on the user's phenotype is generated or collected. An applicable engine for managing production of content to facilitate behavior changes in users, such as the content production management engines described in this paper, can generate or collect content data for producing content to facilitate a behavior change in a user based on application of a behavior change model selected based on the user's phenotype. For example, content can be generated or collected according to behavior change communication instructions generated though application of BCTs of a behavior change model according to a context associated with a user.

The flowchart 1000 continues to module 1004, where content production instructions are added to the content data for use in controlling production of the content using the content data. An applicable engine for managing a form in which content is produced, such as the content form production management engines described in this paper, can add content production instructions to the content data for use in controlling production of the content using the content data. Content production instructions can be generated and added to the content data according to behavior change communication instructions. Additionally, content production instructions can be generated and added to the content data according to a user profile maintained for the user.

The flowchart 1000 continues to module 1006, where the content data is provided to an applicable device for use in producing the content at the device to facilitate the behavior change in the user. For example, the content data can be provided to a behavior change nudge device for use in reproducing content using the content data to facilitate the behavior change in the user. An applicable engine for communicating for purposes of facilitating behavior changes, such as the content production communication engines described in this paper, can provide the content data for use in producing the content to facilitate the behavior change in the user.

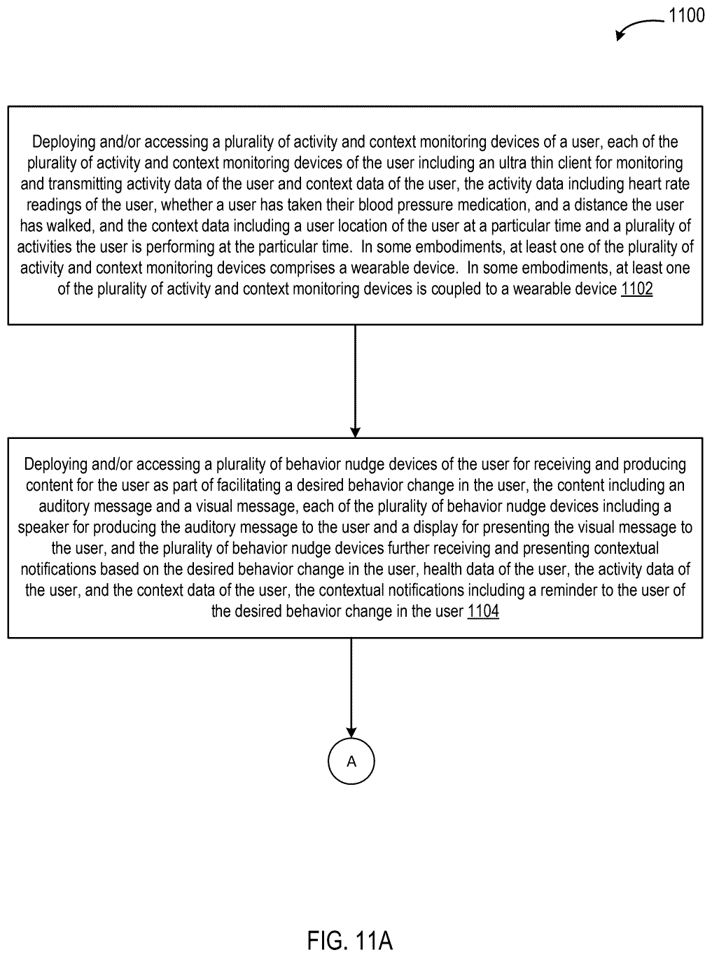

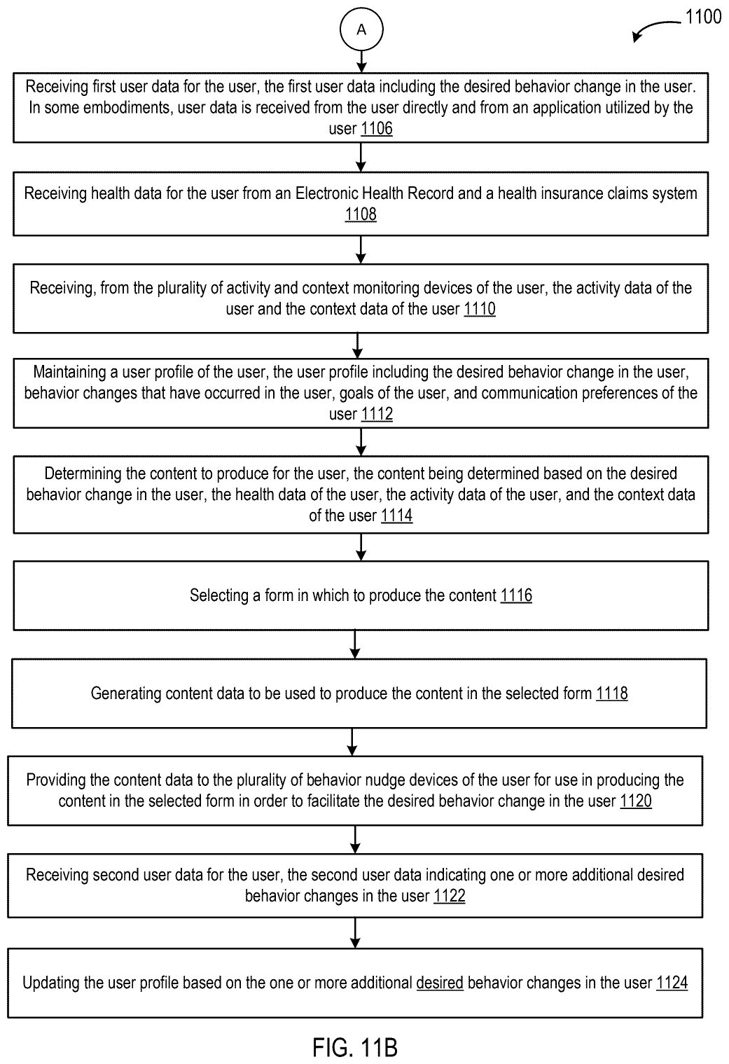

FIGS. 11A and 11B depict a flowchart 1100 of an example of a method for facilitating behavior changes in a user through content generation, activity and context monitoring devices, and nudge devices. In this and other flowcharts and/or sequence diagrams, the flowchart illustrates by way of example a sequence of modules. It should be understood that some or all of the modules may be repeated, reorganized for parallel execution, and/or reordered, as applicable. Moreover, some modules that could have been included may have been removed to avoid providing too much information for the sake of clarity and some modules that were included could be removed but may have been included for the sake of illustrative clarity.

In module 1102, a computing system deploys and/or accesses a plurality of activity and context monitoring devices of a user, each of the plurality of activity and context monitoring devices of the user including an ultra thin client for monitoring and transmitting activity data of the user and context data of the user, the activity data including heart rate readings of the user, whether a user has taken their blood pressure medication, and a distance the user has walked, and the context data including a user location of the user at a particular time and a plurality of activities the user is performing at the particular time. In some embodiments, at least one of the plurality of activity and context monitoring devices comprises a wearable device. In some embodiments, at least one of the plurality of activity and context monitoring devices is coupled to a wearable device.

In module 1104, the computing system deploys and/or accesses a plurality of behavior nudge devices of the user for receiving and producing content for the user as part of facilitating a desired behavior change in the user, the content including an auditory message and a visual message, each of the plurality of behavior nudge devices including a speaker for producing the auditory message to the user and a display for presenting the visual message to the user, and the plurality of behavior nudge devices further receiving and presenting contextual notifications based on the desired behavior change in the user, health data of the user, the activity data of the user, and the context data of the user, the contextual notifications including a reminder to the user of the desired behavior change in the user.

In module 1106, the computing system receives first user data for the user, the first user data including the desired behavior change in the user. In some embodiments, user data is received from the user directly and from an application utilized by the user.

In module 1108, the computing system receives health data for the user from an Electronic Health Record and a health insurance claims system.

In module 1110, the computing system receives, from the plurality of activity and context monitoring devices of the user, the activity data of the user and the context data of the user.

In step 1112, the computing system maintains a user profile of the user, the user profile including the desired behavior change in the user, behavior changes that have occurred in the user, goals of the user, and communication preferences of the user.

In step 1114, the computing system determines the content to produce for the user, the content being determined based on the desired behavior change in the user, the health data of the user, the activity data of the user, and the context data of the user.

In step 1116, the computing system selects a form in which to produce the content.

In step 1118, the computing system generates content data to be used to produce the content in the selected form.

In step 1120, the computing system provides the content data to the plurality of behavior nudge devices of the user for use in producing the content in the selected form in order to facilitate the desired behavior change in the user.

In step 1122, the computing system receives second user data for the user, the second user data indicating one or more additional desired behavior changes in the user.

In step 1124, the computing system updates the user profile based on the one or more additional desired behavior changes in the user.

In some embodiments, the desired behaviour change in the user is a reduction in calories consumed, and the recommendation rules comprise a recommendation of a restaurant. In some embodiments, the desired behaviour change in the user is an increase in physical fitness, and the recommendation rules comprise a recommendation of a gym.

These and other examples provided in this paper are intended to illustrate but not necessarily to limit the described implementation. As used herein, the term “implementation” means an implementation that serves to illustrate by way of example but not limitation. The techniques described in the preceding text and figures can be mixed and matched as circumstances demand to produce alternative implementations.

We claim:

1. A system comprising:
a plurality of activity and context monitoring devices of a user, each of the plurality of activity and context monitoring devices of the user including an ultra thin client for monitoring and transmitting activity data of the user and context data of the user, the activity data including heart rate readings of the user, whether a user has taken their blood pressure medication, and a distance the user has walked, and the context data including a user location of the user at a particular time and a plurality of activities the user is performing at the particular time;
a plurality of behavior nudge devices of the user for receiving and producing content for the user as part of facilitating a desired behavior change in the user, the content including an auditory message and a visual message, each of the plurality of behavior nudge devices including a speaker for producing the auditory message to the user and a display for presenting the visual message to the user, and the plurality of behavior nudge devices further receiving and presenting contextual notifications based on the desired behavior change in the user, health data of the user, the activity data of the user, and the context data of the user, the contextual notifications including a reminder to the user of the desired behavior change in the user;
one or more processors; and
memory storing instructions that, when executed by the one or more processors, cause the system to perform:
receiving first user data for the user, the first user data including the desired behavior change in the user;
receiving the health data for the user from an Electronic Health Record and a health insurance claims system;
receiving, from the plurality of activity and context monitoring devices of the user, the activity data of the user and the context data of the user;
maintaining a user profile of the user, the user profile including the desired behavior change in the user, behavior changes that have occurred in the user, goals of the user, and communication preferences of the user;
determining the content to produce for the user, the content being determined based on the desired behavior change in the user, the health data of the user, the activity data of the user, and the context data of the user;
selecting a form in which to produce the content;
generating content data to be used to produce the content in the selected form;
providing the content data to the plurality of behavior nudge devices of the user for use in producing the content in the selected form in order to facilitate the desired behavior change in the user;
receiving second user data for the user, the second user data indicating one or more additional desired behavior changes in the user; and
updating the user profile based on the one or more additional desired behavior changes in the user.

2. The system of claim 1, wherein the user data is received from the user directly and from an application utilized by the user.

3. The system of claim 1, wherein at least one of the plurality of activity and context monitoring devices comprises a wearable device.

4. The system of claim 1, wherein at least one of the plurality of activity and context monitoring devices is coupled to a wearable device.

5. The system of claim 1, wherein the instructions, when executed by the one or more processors, further cause the system to perform:
providing an interface to a caregiver of the user through which the caregiver can view the activity data of the user, the health data of the user, and the context data of the user; and
receiving from the caregiver, via the interface, motivation rules and recommendation rules to facilitate the behavior change in the user, the motivation rules and recommendation rules being based on the desired behavior change in the user, the activity data of the user, the health data of the user, and the context data of the user.

6. The system of claim 5, wherein the desired behavior change in the user is a reduction in calories consumed, and the recommendation rules comprise a recommendation of a restaurant.

7. The system of claim 5, wherein the desired behavior change in the user is an increase in physical fitness, and the recommendation rules comprise a recommendation of a gym.
